SEQUENCE LISTING (36 sequences)

SEQ ID NO: 1 - protein, 22 aa, Artificial Sequence
  Synthetic peptide
  MISC_FEATURE (1)..(5): Xaa at positions 1 to 5 may be any amino acid
  MISC_FEATURE (8)..(8): Xaa at position 8 may be any amino acid
  MISC_FEATURE (14)..(14): Xaa at position 14 may be any amino acid
  MISC_FEATURE (16)..(16): Xaa at position 16 may be any amino acid
XXXXXGVXAKAGVXNXFKSESY

SEQ ID NO: 2 - protein, 22 aa, Artificial Sequence
  Synthetic peptide
  MISC_FEATURE (1)..(1): Xaa at position 1 is any amino acid or is absent
  MISC_FEATURE (2)..(2): Xaa at position 2 is any amino acid or is absent
  MISC_FEATURE (3)..(3): Xaa at position 3 is any amino acid or is absent
  MISC_FEATURE (4)..(4): Xaa at position 4 is any amino acid or is absent
  MISC_FEATURE (5)..(5): Xaa at position 5 is any amino acid or is absent
  MISC_FEATURE (8)..(8): Xaa at position 8 is any amino acid or is absent
  MISC_FEATURE (14)..(14): Xaa at position 14 is any amino acid or is absent
  MISC_FEATURE (16)..(16): Xaa at position 16 is any amino acid or is absent
XXXXXGVXAKAGVXNXFKSESY

SEQ ID NO: 3 - protein, 24 aa, Artificial Sequence
  Synthetic peptide
  MISC_FEATURE (3)..(3): Xaa at position 3 may be any amino acid
  MISC_FEATURE (7)..(7): Xaa at position 7 may be any amino acid
  MISC_FEATURE (13)..(13): Xaa at position 13 may be any amino acid
  MISC_FEATURE (22)..(22): Xaa at position 22 may be Gln, Pro, Tyr, Ile or Leu
  MISC_FEATURE (23)..(23): Xaa at position 23 may be Gln, Pro, Tyr, Ile or Leu
YKXLRRXAPRWDXPLRDPALRXXL

SEQ ID NO: 4 - protein, 22 aa, Artificial Sequence
  Synthetic peptide
  MISC_FEATURE (3)..(3): Xaa at position 3 may be any amino acid
  MISC_FEATURE (7)..(7): Xaa at position 7 may be any amino acid or is absent
  MISC_FEATURE (8)..(8): Xaa at position 8 may be any amino acid or is absent
  MISC_FEATURE (9)..(9): Xaa at position 9 may be any amino acid or is absent
  MISC_FEATURE (10)..(10): Xaa at position 10 may be any amino acid or is absent
  MISC_FEATURE (11)..(11): Xaa at position 11 may be any amino acid or is absent
  MISC_FEATURE (20)..(20): Xaa at position 20 may be Gln, Pro, Tyr, Ile or Leu
  MISC_FEATURE (21)..(21): Xaa at position 21 may be Gln, Pro, Tyr, Ile or Leu
YKXLRRXXXXXPLRDPALRXXL

SEQ ID NO: 5 - protein, 22 aa, Artificial Sequence
  Synthetic peptide
IKLSGGVQAKAGVINMDKSESM

SEQ ID NO: 6 - protein, 22 aa, Artificial Sequence
  Synthetic peptide
IKLSGGVQAKAGVINMFKSESY

SEQ ID NO: 7 - protein, 23 aa, Artificial Sequence
  Synthetic peptide
IKLSGGVQAKAGVINMFKSESYK

SEQ ID NO: 8 - protein, 17 aa, Artificial Sequence
  GVQAKAGVINMFKSESY
GVQAKAGVINMFKSESY

SEQ ID NO: 9 - protein, 17 aa, Artificial Sequence
  Synthetic peptide
GVRAKAGVRNMFKSESY

SEQ ID NO: 10 - protein, 17 aa, Artificial Sequence
  Synthetic peptide
  MOD_RES (11)..(11): Nle
GVRAKAGVRNXFKSESY

SEQ ID NO: 11 - protein, 24 aa, Artificial Sequence
  Synthetic peptide
YKSLRRKAPRWDAPLRDPALRQLL

SEQ ID NO: 12 - protein, 24 aa, Artificial Sequence
  Synthetic peptide
YKSLRRKAPRWDAYLRDPALRQLL

SEQ ID NO: 13 - protein, 24 aa, Artificial Sequence
  Synthetic peptide
YKSLRRKAPRWDAYLRDPALRPLL

SEQ ID NO: 14 - protein, 17 aa, Artificial Sequence
  Synthetic peptide
  MOD_RES (1)..(1): ACETYLATION
GVRAKAGVRNMFKSESY

SEQ ID NO: 15 - protein, 17 aa, Artificial Sequence
  Synthetic peptide
  MOD_RES (1)..(1): ACETYLATION
  MOD_RES (11)..(11): Nle
GVRAKAGVRNXFKSESY

SEQ ID NO: 16 - protein, 24 aa, Artificial Sequence
  Synthetic peptide
  MOD_RES (1)..(1): ACETYLATION
YKSLRRKAPRWDAPLRDPALRQLL

SEQ ID NO: 17 - protein, 24 aa, Artificial Sequence
  Synthetic peptide
  MOD_RES (1)..(1): ACETYLATION
YKSLRRKAPRWDAYLRDPALRQLL

SEQ ID NO: 18 - protein, 24 aa, Artificial Sequence
  Synthetic peptide
YKSLRRKAPRWDAYLRDPALRPLL

SEQ ID NO: 19 - protein, 5 aa, Artificial Sequence
  Synthetic linker
  MISC_FEATURE (1)..(1): Xaa is Thr or Ser
  MISC_FEATURE (1)..(4): No more than two of the Xaa at positions 1 to 4 are Thr
  MISC_FEATURE (2)..(2): Xaa is Thr or Ser
  MISC_FEATURE (3)..(3): Xaa is Thr or Ser
  MISC_FEATURE (4)..(4): Xaa is Thr or Ser
XXXXG

SEQ ID NO: 20 - protein, 5 aa, Artificial Sequence
  Synthetic linker
SSSSG

SEQ ID NO: 21 - protein, 5 aa, Artificial Sequence
  Synthetic linker
PAPAP

SEQ ID NO: 22 - protein, 7 aa, Artificial Sequence
  Synthetic linker
AEAAAKA

SEQ ID NO: 23 - protein, 17 aa, Artificial Sequence
  Synthetic conjugate
  MISC_FEATURE (5)..(5): An antitumor agent is attached to the side chain of the Lys
  MOD_RES (11)..(11): Nle
  MISC_FEATURE (13)..(13): An antitumor agent is attached to the side chain of the Lys
GVRAKAGVRNXFKSESY

SEQ ID NO: 24 - protein, 17 aa, Artificial Sequence
  Synthetic conjugate
  MOD_RES (1)..(1): ACETYLATION
  MISC_FEATURE (5)..(5): An antitumor agent is attached to the side chain of the Lys
  MOD_RES (11)..(11): Nle
  MISC_FEATURE (13)..(13): An antitumor agent is attached to the side chain of the Lys
GVRAKAGVRNXFKSESY

SEQ ID NO: 25 - protein, 17 aa, Artificial Sequence
  Synthetic conjugate
  MISC_FEATURE (5)..(5): A curcumin molecule is attached to the side chain of the Lys
  MOD_RES (11)..(11): Nle
  MISC_FEATURE (13)..(13): A curcumin molecule is attached to the side chain of the Lys
GVRAKAGVRNXFKSESY

SEQ ID NO: 26 - protein, 24 aa, Artificial Sequence
  Synthetic conjugate
  MISC_FEATURE (2)..(2): A curcumin molecule is attached to the side chain of the Lys
  MISC_FEATURE (7)..(7): A curcumin molecule is attached to the side chain of the Lys
YKSLRRKAPRWDAPLRDPALRQLL

SEQ ID NO: 27 - protein, 17 aa, Artificial Sequence
  Synthetic conjugate
  MOD_RES (1)..(1): ACETYLATION
  MOD_RES (5)..(5): A curcumin molecule is attached to the side chain of the Lys
  MOD_RES (11)..(11): Nle
  MOD_RES (13)..(13): A curcumin molecule is attached to the side chain of the Lys
GVRAKAGVRNXFKSESY

SEQ ID NO: 28 - protein, 24 aa, Artificial Sequence
  Synthetic conjugate
  MOD_RES (1)..(1): ACETYLATION
  MISC_FEATURE (2)..(2): A curcumin molecule is attached to the side chain of the Lys
  MISC_FEATURE (7)..(7): A curcumin molecule is attached to the side chain of the Lys
YKSLRRKAPRWDAPLRDPALRQLL

SEQ ID NO: 29 - protein, 17 aa, Artificial Sequence
  Synthetic conjugate
  MISC_FEATURE (5)..(5): A docetaxel molecule is attached to the side chain of the Lys
  MOD_RES (11)..(11): Nle
  MISC_FEATURE (13)..(13): A docetaxel molecule is attached to the side chain of the Lys
GVRAKAGVRNXFKSESY

SEQ ID NO: 30 - protein, 17 aa, Artificial Sequence
  Synthetic conjugate
  MOD_RES (1)..(1): ACETYLATION
  MISC_FEATURE (5)..(5): A docetaxel molecule is attached to the side chain of the Lys
  MOD_RES (11)..(11): Nle
  MISC_FEATURE (13)..(13): A docetaxel molecule is attached to the side chain of the Lys
GVRAKAGVRNXFKSESY

SEQ ID NO: 31 - protein, 18 aa, Artificial Sequence
  Synthetic peptide
  MOD_RES (11)..(11): Nle
GVRAKAGVRNXFKSESYC

SEQ ID NO: 32 - protein, 18 aa, Artificial Sequence
  Synthetic peptide
  MOD_RES (1)..(1): ACETYLATION
  MOD_RES (11)..(11): Nle
GVRAKAGVRNXFKSESYC

SEQ ID NO: 33 - protein, 17 aa, Artificial Sequence
  Synthetic conjugate
  MISC_FEATURE (5)..(5): A doxorubicin molecule is attached to the side chain of the Lys
  MOD_RES (11)..(11): Nle
  MISC_FEATURE (13)..(13): A doxorubicin molecule is attached to the side chain of the Lys
GVRAKAGVRNXFKSESY

SEQ ID NO: 34 - protein, 17 aa, Artificial Sequence
  Synthetic conjugate
  MOD_RES (1)..(1): ACETYLATION
  MISC_FEATURE (5)..(5): A doxorubicin molecule is attached to the side chain of the Lys
  MOD_RES (11)..(11): Nle
  MISC_FEATURE (13)..(13): A doxorubicin molecule is attached to the side chain of the Lys
GVRAKAGVRNXFKSESY

SEQ ID NO: 35 - protein, 18 aa, Artificial Sequence
  Synthetic conjugate
  MOD_RES (11)..(11): Nle
  MISC_FEATURE (18)..(18): An aldoxorubicin molecule is attached to the side chain of the Cys residue
GVRAKAGVRNXFKSESYC

SEQ ID NO: 36 - protein, 18 aa, Artificial Sequence
  Synthetic conjugate
  MOD_RES (1)..(1): ACETYLATION
  MOD_RES (11)..(11): Nle
  MISC_FEATURE (18)..(18): An aldoxorubicin molecule is attached to the side chain of the Cys
GVRAKAGVRNXFKSESYC

(19) United States
(12) Patent Application Publication
(10) Pub. No.: US 2024/0156971 A1
(43) Pub. Date: May 16, 2024

(54) METHODS AND SORTILIN BINDING CONJUGATE COMPOUNDS FOR TARGETING CANCER STEM CELLS

(71) Applicants: TRANSFERT PLUS, SOCIÉTÉ EN COMMANDITE, Quebec (CA); THERATECHNOLOGIES INC., Montréal (CA)

(72) Inventors: Richard Beliveau, Verdun (CA); Michel Demeule, Beaconsfield (CA); Borhane Annabi, Brossard (CA); Cyndia Charfi, La Prairie (CA); Alain Larocque, Saint-Laurent (CA); Jean-Christophe Currie, Repentigny (CA); Alain Zgheib, Montreal (CA); Christian Marsolais, Mont-Royal (CA)

(73) Assignees: TRANSFERT PLUS, SOCIÉTÉ EN COMMANDITE, Quebec (CA); THERATECHNOLOGIES INC., Montreal (CA)

(21) Appl. No.: 18/278,821

(22) PCT Filed: Feb. 24, 2022
(86) PCT No.: PCT/CA2022/050263

§ 371 Date: Aug. 24, 2023

Related U.S. Application Data
(60) Provisional application No. 63/200,284, filed on Feb. 26, 2021.
(60) Provisional application No. 63/264,105, filed on Nov. 16, 2021.

(51) Int. Cl. A61K 47/64; A61K 31/337; A61K 45/06; A61P 35/00; C07K 7/08; C07K 14/705

(52) U.S. Cl. CPC A61K 47/64; A61K 31/337; A61K 45/06; A61P 35/00; C07K 7/08; C07K 14/705

(57) ABSTRACT

The present application relates to methods and uses of conjugates comprising antitumor agents (e.g., chemotherapeutic agents) conjugated to peptide compounds targeting Sortilin-expressing cancer stem cells (CSCs), in embodiments for the treatment of poor prognosis cancers refractory to standard antitumor therapies associated the presence of Sortilin-expressing CSCs, and for preventing or treating cancer relapse or recurrence.

### CROSS-REFERENCE TO RELATED APPLICATIONS

The present claims the benefit of U.S. provisional patent application Ser. No. 63/200,284 filed Feb. 26, 2021, and of U.S. provisional patent application Ser. No. 63/264,105 filed Nov. 16, 2021, which are incorporated herein by reference.

### SEQUENCE LISTING

This application contains a Sequence Listing in computer readable form entitled “G11718-00423_SeqList_ST25.txt”, created on Feb. 24, 2022 and having a size of about 17 kB. The computer readable form is incorporated herein by reference.

### TECHNICAL FIELD

The present disclosure generally relates to the field of oncology, and more particularly to compounds and methods and uses thereof for targeting cancer stem cells (CSCs).

### BACKGROUND ART

According to a recent World Health Organization report, 8.2 million patients died from cancer in 2012. Cancer is therefore a continuously growing health problem in both developing and developed countries. It has also been estimated that the number of annual cancer cases will increase within the next two decades. The common general treatments for cancer are surgery, endocrine therapy, chemotherapy, immunotherapy and radiotherapy.

Because of all these treatments, the incidence rate of cancer has been stable in women and has declined slightly in men during recent years (2006-2015), and the cancer death rate (2007-2016) also declined. However, traditional cancer treatment methods are effective only for some malignant tumors. The main reasons for the failure of cancer treatment are metastasis, recurrence, heterogeneity, resistance to chemotherapy and radiotherapy, and avoidance of immunological surveillance. Nearly all current cancer treatments focus on de-bulking tumors without targeting the most dangerous cells in the tumor: cancer stem cells (CSCs). CSCs are responsible for the spread of cancer cells throughout the body, the growth of tumors, cancer's resistance to chemotherapy, and the recurrence of tumors after treatment or surgical removal, notably through their ability to arrest in the G0 phase, giving rise to new tumors. Because current treatments do not target the CSC population, they frequently lead to the rise of resistant tumors and continued cancer spread. Therefore, CSCs could be considered the most promising targets for cancer treatment.

There is thus a need for the development of strategies for the treatment of cancer, and more particularly cancers associated with CSCs, for example to overcome tumor resistance to cancer therapy, and prevent/treat cancer relapse and improve survival in cancer patients.

The present description refers to a number of documents, the content of which is herein incorporated by reference in their entirety.

### SUMMARY

The present disclosure to products (e.g., compounds), and methods and uses thereof for targeting cancer stem cells, and related uses.

In various aspects and embodiments, the present disclosure provides the following items:
1. A method for treating a cancer comprising Sortilin-expressing cancer stem cells (CSCs) in a subject comprising administering to the subject an effective amount of a conjugate compound or a pharmaceutically acceptable salt thereof, wherein the conjugate compound has the formula of A-(B)n, wherein
A is a peptide compound of 30 residues or less comprising an amino acid sequence having at least 60% sequence identity to one of the sequences of formulas (I)-(XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)_{N}PLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid,
optionally the peptide compound is cyclic,
B is at least one antitumor agent, wherein B is connected to A directly or via a linker.
2. The method of item 1, wherein the peptide compound has an amino acid sequence having at least 90% sequence identity to one of the sequences of formulas (I)-(XIII).
3. The method of item 1 or 2, wherein the peptide compound comprises one of the sequences of formulas (I)-(XIII), and further comprises 1 to 3 additional amino acids at its amino- and/or carboxy-terminal end.
4. The method of item 3, wherein the peptide compound comprises a cysteine residue at its amino- and/or carboxy-terminal end.
5. The method of item 1, wherein the peptide compound is represented by formula (I) and has an amino acid sequence consisting of the amino acid sequence of SEQ ID NO: 1 or 2.
6. The method of item 1, wherein the peptide compound is represented by formula (III) and has an amino acid sequence consisting of the amino acid sequence of SEQ ID NO: 3 or 4.
7. The method of item 1, wherein the peptide compound is represented by formula (V) and has an amino acid sequence consisting of the amino acid sequence of SEQ ID NO: 5.
8. The method of item 1, wherein the peptide compound is represented by formula (VI) and has an amino acid sequence consisting of the amino acid sequence of SEQ ID NO: 6.
9. The method of item 1, wherein the peptide compound is represented by formula (VII) and has an amino acid sequence consisting of the amino acid sequence of SEQ ID NO: 7.
10. The method of item 1, wherein the peptide compound is represented by formula (VIII) and has an amino acid sequence consisting of the amino acid sequence of SEQ ID NO: 8.
11. The method of item 1, wherein the peptide compound is represented by formula (IX) and has an amino acid sequence consisting of the amino acid sequence of SEQ ID NO: 9.
12. The method of item 1, wherein the peptide compound is represented by formula (X) and has an amino acid sequence consisting of the amino acid sequence of SEQ ID NO: 10.
13. The method of item 1, wherein the peptide compound is represented by formula (XI) and has an amino acid sequence consisting of the amino acid sequence of SEQ ID NO: 11.
14. The method of item 1, wherein the peptide compound is represented by formula (XII) and has an amino acid sequence consisting of the amino acid sequence of SEQ ID NO: 12.
15. The method of item 1, wherein the peptide compound is represented by formula (XIII) and has an amino acid sequence consisting of the amino acid sequence of SEQ ID NO: 13.
16. The method of any one of items 1 to 15, wherein the peptide compound comprises at least one modifying group at its amino- and/or carboxy-terminal end.
17. The method of item 16, wherein the at least one modifying group is acetyl or succinyl.
18. The method of item 1, wherein the peptide compound is represented by Formula (XXXVIII), (XXXIX), (XL), (XLI) or (XLII):

(XXXVIII)
(SEQ ID NO: 14)
Acetyl-GVRAKAGVRNMFKSESY
(XXXIX)
(SEQ ID NO: 15)
Acetyl-GVRAKAGVRN(NIe)FKSESY
(XL)
(SEQ ID NO: 16)
Acetyl-YKSLRRKAPRWDAPLRDPALRQLL
(XLI)
(SEQ ID NO: 17)
Acetyl-YKSLRRKAPRWDAYLRDPALRQLL
(XLII)
(SEQ ID NO: 18)
Acetyl-YKSLRRKAPRWDAYLRDPALRPLL.

19. The method of any one of items 1 to 18, wherein B is connected to A at a free amine of said peptide compound, at an N-terminal position of said peptide compound, at a free —SH of said peptide compound, and/or at a free carboxyl of said peptide compound.
20. The method of any one of items 1 to 19, wherein B is connected to A via a linker.
21. The method of any one of items 1 to 20, wherein the conjugate is represented by formula (LIII) or (LIV):

(LIII)
(SEQ ID NO: 23)
GVRAK(Z¹)AGVRN(Nle)FK(Z²)SESY;
(LIV)
(SEQ ID NO: 24)
Acetyl-GVRAK(Z¹)AGVRN(Nle)FK(Z²)SESY;

wherein Z₁ and Z₂ are each independently an antitumor agent attached to the lysine (K) residues.
22. The method of any one of items 1 to 21, wherein the antitumor agent is a radionuclide or a chemotherapeutic agent.
23. The method of item 22, wherein the chemotherapeutic agent is a taxane.
24. The method of item 23, wherein the chemotherapeutic agent is docetaxel.
25. A method for preventing or treating a cancer relapse or recurrence associated with the presence of Sortilin-expressing cancer stem cells (CSCs) in a subject, the method comprising administering to the subject an effective amount of the conjugate or salt thereof defined in any one of items 1 to 24.
26. A method for treating a poor prognosis cancer associated with the presence of Sortilin-expressing cancer stem cells (CSCs) in a subject, the method comprising administering to the subject an effective amount of the conjugate or salt thereof defined in any one of items 1 to 24.
27. A method for treating an unresectable, chemotherapy- and radiotherapy-resistant cancer associated with the presence of Sortilin-expressing cancer stem cells (CSCs) in a subject, the method comprising administering to the subject an effective amount of the conjugate or salt thereof defined in any one of items 1 to 24.
28. The method of any one of items 1 to 27, wherein the CSCs express at least one multidrug resistance (MDR) protein.
29. The method of item 28, wherein the CSCs express MDR1 and/or ABCB5.
30. The method of any one of items 1 to 29, wherein the cancer is breast cancer, urogenital cancer, pancreatic cancer, lung cancer, thyroid cancer, kidney cancer, gastrointestinal tract cancer, neuroendocrine tumors, skin cancer, brain cancer and leukemia.
31. The method of item 30, wherein the urogenital cancer is ovarian cancer, prostate cancer, endometrial cancer or testis cancer.
32. The method of item 30, wherein the breast cancer is invasive ductal carcinoma (IDC) or triple negative breast cancer (TNBC).
33. The method of item 30, wherein the kidney cancer is renal cell carcinoma (RCC).
34. The method of item 30, wherein the gastrointestinal tract cancer is colorectal cancer.
35. The method of item 30, wherein the skin cancer is melanoma.
36. The method of item 30, wherein the brain cancer is glioma.
37. The method of item 30, wherein the leukemia is B cell Chronic Lymphocytic Leukemia (B-CLL).
38. The method of item 30, wherein the cancer is breast cancer, ovarian cancer or pancreatic cancer.
39. The method of any one of items 1 to 38, wherein the administration of the conjugate or salt thereof inhibits migration of the CSCs in the subject.
40. The method of any one of items 1 to 39, wherein the method further comprises administering one or more additional active agents or therapies to the subject.
41. The method of item 40, where the one or more additional active agents or therapies comprise radiotherapy, surgery, chemotherapeutic agents, immunotherapies, checkpoint inhibitors, and/or cell-based therapies.
42. Use of the conjugate or salt thereof defined in any one of items 1 to 24 for the preparation of a medicament for treating a cancer comprising Sortilin-expressing cancer stem cells (CSCs) in a subject.
43. Use of the conjugate or salt thereof defined in any one of items 1 to 24 for the preparation of a medicament for preventing or treating a cancer relapse or recurrence associated with the presence of Sortilin-expressing cancer stem cells (CSCs) in a subject.
44. Use of the conjugate or salt thereof defined in any one of items 1 to 24 for the preparation of a medicament for treating a poor prognosis cancer associated with the presence of Sortilin-expressing cancer stem cells (CSCs) in a subject.
45. Use of the conjugate or salt thereof defined in any one of items 1 to 24 for the preparation of a medicament for treating an unresectable, chemotherapy- and radiotherapy-resistant cancer associated with the presence of Sortilin-expressing cancer stem cells (CSCs) in a subject.
46. Use of the conjugate or salt thereof defined in any one of items 1 to 24 for treating a cancer comprising Sortilin-expressing cancer stem cells (CSCs) in a subject.
47. Use of the conjugate or salt thereof defined in any one of items 1 to 24 for preventing or treating a cancer relapse or recurrence associated with the presence of Sortilin-expressing cancer stem cells (CSCs) in a subject.
48. Use of the conjugate or salt thereof defined in any one of items 1 to 24 for treating a poor prognosis cancer associated with the presence of Sortilin-expressing cancer stem cells (CSCs) in a subject.
49. Use of the conjugate or salt thereof defined in any one of items 1 to 24 for treating an unresectable, chemotherapy- and radiotherapy-resistant cancer associated with the presence of Sortilin-expressing cancer stem cells (CSCs) in a subject.
50. The use of any one of items 42 to 49, wherein the CSCs express at least one multidrug resistance (MDR) protein.
51. The use of item 50, wherein the CSCs express MDR1 and/or ABCB5.
52. The use of any one of items 42 to 51, wherein the cancer is breast cancer, urogenital cancer, pancreatic cancer, lung cancer, thyroid cancer, kidney cancer, gastrointestinal tract cancer, neuroendocrine tumors, skin cancer, brain cancer and leukemia.
53. The use of item 52, wherein the urogenital cancer is ovarian cancer, prostate cancer, endometrial cancer or testis cancer.
54. The use of item 52, wherein the breast cancer is invasive ductal carcinoma (IDC) or triple negative breast cancer (TNBC).
55. The use of item 52, wherein the kidney cancer is renal cell carcinoma (RCC).
56. The use of item 52, wherein the gastrointestinal tract cancer is colorectal cancer.
57. The use of item 52, wherein the skin cancer is melanoma.
58. The use of item 52, wherein the brain cancer is glioma.
59. The use of item 52, wherein the leukemia is B cell Chronic Lymphocytic Leukemia (B-CLL).
60. The use of item 52, wherein the cancer is breast cancer, ovarian cancer or pancreatic cancer.
61. The use of any one of items 42 to 60, wherein the medicament inhibits migration of the CSCs in the subject.
62. The use of any one of items 42 to 61, wherein the medicament is for use with one or more additional active agents or therapies.
63. The use of item 62, where the one or more additional active agents or therapies comprise radiotherapy, surgery, chemotherapeutic agents, immunotherapies, checkpoint inhibitors, and/or cell-based therapies.
64. The conjugate or salt thereof as defined in any one of items 1 to 24 for use in treating a cancer comprising Sortilin-expressing cancer stem cells (CSCs) in a subject.
65. The conjugate or salt thereof as defined in any one of items 1 to 24 for use in preventing or treating a cancer relapse or recurrence associated with the presence of Sortilin-expressing cancer stem cells (CSCs) in a subject.
66. The conjugate or salt thereof as defined in any one of items 1 to 24 for use in treating a poor prognosis cancer associated with the presence of Sortilin-expressing cancer stem cells (CSCs) in a subject.
67. The conjugate or salt thereof as defined in any one of items 1 to 24 for use in treating an unresectable, chemotherapy- and radiotherapy-resistant cancer associated with the presence of Sortilin-expressing cancer stem cells (CSCs) in a subject.
68. The conjugate or salt thereof for use according to any one of items 64 to 67, wherein the CSCs express at least one multidrug resistance (MDR) protein.
69. The conjugate or salt thereof for use according to item 68, wherein the CSCs express MDR1 and/or ABCB5.
70. The conjugate or salt thereof for use according to any one of items 65 to 69, wherein the cancer is breast cancer, urogenital cancer, pancreatic cancer, lung cancer, thyroid cancer, kidney cancer, gastrointestinal tract cancer, neuroendocrine tumors, skin cancer, brain cancer and leukemia.
71. The conjugate or salt thereof for use according to item 70, wherein the urogenital cancer is ovarian cancer, prostate cancer, endometrial cancer or testis cancer.
72. The conjugate or salt thereof for use according to item 70, wherein the breast cancer is invasive ductal carcinoma (IDC) or triple negative breast cancer (TNBC).
73. The conjugate or salt thereof for use according to item 70, wherein the kidney cancer is renal cell carcinoma (RCC).
74. The conjugate or salt thereof for use according to item 70, wherein the gastrointestinal tract cancer is colorectal cancer.
75. The conjugate or salt thereof for use according to item 70, wherein the skin cancer is melanoma.
76. The conjugate or salt thereof for use according to item 70, wherein the brain cancer is glioma.
77. The conjugate or salt thereof for use according to item 70, wherein the leukemia is B cell Chronic Lymphocytic Leukemia (B-CLL).
78. The conjugate or salt thereof for use according to item 70, wherein the cancer is breast cancer, ovarian cancer or pancreatic cancer.
79. The conjugate or salt thereof for use according to any one of items 64 to 78, wherein the conjugate or salt thereof inhibits migration of the CSCs in the subject.
380. The conjugate or salt thereof for use according to any one of items 64 to 79, wherein the conjugate or salt thereof is for use with one or more additional active agents or therapies.
81. The conjugate or salt thereof according to item 80, where the one or more additional active agents or therapies comprise radiotherapy, surgery, chemotherapeutic agents, immunotherapies, checkpoint inhibitors, and/or cell-based therapies.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A depicts an image of a Western blot showing the expression of Sortilin (SORT1) by human breast triple-negative MDA-MB-231 tumor cells and breast cancer stem cells (hBCSCs). Human triple-negative BCSCs and MDA-MB-231/luc cell homogenates (20 μg protein) were separated on a polyacrylamide gel and electrotransferred to a PVDF membrane. Sortilin was detected with two different antibodies (SORT1 #1: BD Biosciences, SORT1 #2: Abcam) on the membrane by Western blotting.

FIG. 1B is a graph showing the internalization of TH19P01-Alexa Fluor 488 by hBCSCs. hBCSCs were exposed to media to which was added 200 nM fluorescently labelled TH19P01 or vehicle alone. The fluorescence contained within the cells was measured by flow cytometry after cells were incubated for 2 hours, washed and trypsinized. The data shown represent means±SEM, n=3, each performed in duplicate. Statistical comparison was done by t-test, significance assumed for p<0.05. Triple asterisks denote p<0.001.

FIGS. 2A and 2B are graphs showing the internalization of TH19P01-Alexa Fluor 488 by hBCSCs in the presence of Sortilin ligands (FIG. 2A) and upon siRNA-mediated SORT1 silencing (FIG. 2B). FIG. 2A: Control data refers to cells which were incubated in the presence of TH19P01-Alexa Fluor 488 without any competing ligands. The uptake of fluorescently labeled TH19P01 was also performed in the additional presence of Sortilin ligands: 10 μM neurotensin (NT), 50 μM non-fluorescent TH19P01 or 1 nM progranulin (PGRN). The data was compared by one-way ANOVA followed by Tukey's multiple comparison test against the Control fluorescence; significance was assumed for p<0.05. The data shown represent means±SEM, n=3 assays, performed in duplicate. Double asterisks denote p<0.01 and triple asterisks denote p<0.001. FIG. 2B: Cells were preincubated with siRNA prior to incubation with the fluorescent peptide; the two siRNAs used were a scrambled, control siRNA (siScr) and an siRNA directed against human sortilin mRNA (siSORT1). Statistical significance was assessed with a two-sided t-test with significance preset at p<0.05. Double asterisks indicate p<0.01. The data shown represent means±SD, n=2.

FIG. 2C is a graph showing the uptake of TH19P01-Alexa488 by hOvCSCs in the presence of TH19P01, progranulin (PGRN), KBP201, neurotensin (NT) and cysteine peptides (TH19P01 with N- or C-terminal cysteine added). hOvCSCs were exposed to media to which had been added either 200 nM fluorescently labelled TH19P01 in DMSO, DMSO alone (Vehicle), or 200 nM TH19P01 in DMSO along with a competing ligand (10 μM neurotensin [NT], 50 μM non-fluorescent TH19P01, 50 μM TH20P01 or 1 nM progranulin [PGRN]). The fluorescence contained within the cells was measured by flow cytometry after cells were incubated for 2 hours, washed and trypsinized. Fluorescence associated with Vehicle alone reflects background fluorescence of the system. The data shown represent means±SEM, n=2, each performed in duplicate. Uptake inhibitions compared to cells incubated with TH19P01-Alexa™ 488 alone as shown.

FIG. 2D: Cells were preincubated with siRNA prior to incubation with the fluorescent peptide; the two siRNAs used were a scrambled, control siRNA (siScr) and an siRNA directed against human sortilin mRNA (siSORT1). The data shown represent means±SEM, n=1.

FIG. 3 depicts images of slides bearing hBCSCs after treatment with vehicle (Control, DMSO), docetaxel or TH1902. A scratch (wound healing) assay was performed to assess the migration ability of hBCSCs. Slides bearing cells were scratched, treated for 2 h with vehicle (DMSO), 2 μM docetaxel or 1 μM TH1902 (equivalent in docetaxel content to the docetaxel treatment). Cells were then rinsed and incubated in fresh complete media. Images were acquired at 0, 24 and 48 h after the scratching at a 40× magnification. One representative experiment is shown (n=3).

FIG. 4A is a graph showing the total apoptosis over time in hBCSCs after treatment with vehicle (Control, DMSO), docetaxel or TH1902. hBCSCs were treated in medium containing vehicle (DMSO), 4 μM docetaxel or 2 μM TH1902 for 2 h and then incubated in complete medium for 22 h, 48 h or 72 h. The extent of apoptosis was determined by harvesting the cells and then performing flow cytometry following staining with annexin V-FITC and propidium iodide (PI). Data were obtained from 3 different experiments and are represented as mean+/−SEM; statistical analysis was performed using one-way ANOVA with Dunnett's multiple comparison test (* signifies p<0.05 and *** signifies p<0.001 for difference between TH1902 and control condition).

FIG. 4B depicts confocal microscopy images of hBCSCs after treatment with vehicle (Control, DMSO), docetaxel or TH1902. hBCSCs were treated in medium containing vehicle (DMSO), 4 μM docetaxel or 2 μM TH1902 for 2 h and then incubated in complete medium for 48 h. Treated cells were fixed and DNA was stained with DAPI before imaging by confocal microscopy. Arrow indicates an example of nuclear fragmentation.

FIGS. 4C-D are confocal microscopy images of hBCSCs stained with an anti-α-tubulin antibody after treatment with vehicle (Control, DMSO), docetaxel or TH1902. hBCSCs were treated as described above for FIG. 4B. The effects of docetaxel and TH1902 on tubulin were evaluated in treated cells following fixation and immunostaining with anti-α-tubulin antibody. Cells were then visualized using confocal microscopy. Representative pictures from each condition are displayed as stacked planes (n=1). FIG. 4D corresponds to the samples shown in FIG. 4C but displayed in a single focal plane (middle of cells).

FIGS. 5A-D are flow cytometry plots (FIGS. 5A and 5C) and graphs (FIGS. 5B and 5D) showing the effect of docetaxel or TH1902 on G2/M Cell Cycle Arrest of hBCSCs and MDA-MB-231/luc tumor cells. hBCSCs and MDA-MB-231/luc cells were treated for 2 h with vehicle (DMSO), 4 μM Docetaxel or 2 μM TH1902, then incubated in complete fresh media for 22 h or 48 h. DNA content of each cell line was analyzed by flow cytometry using the FxCycle™ PI/RNase Staining Solution. The experiment was repeated at least three times and representative data are shown in FIG. 5A for hBCSCs and in FIG. 5C for MDA-MB-231/luc cells. FIG. 5A: representative flow cytometry acquisition plots showing the percentage of hBCSCs in G2/M phase under different treatments. FIG. 5B: graph showing the relative frequency of hBCSCs in the G2/M cell cycle phase following the various treatments (x-fold over control). Data are represented as means+/−SEM from 3 different experiments and statistical analysis was performed using one-way ANOVA with Dunnett's multiple comparison test (*** signifies p<0.001 for difference between TH1902 and control condition). FIG. 5C: representative flow cytometry acquisition plots showing the percentage of MDA-MB-231/luc cells in G2/M phase under different treatments. FIG. 5D: graph showing the relative frequency of MDA-MB-231/luc cells in the G2/M cell cycle phase following the various treatments (x-fold over control). Data are represented as means+/−SEM from 2 different experiments.

FIG. 6A depicts an image of a Western blot showing the expression of levels of MDR1 (aka P-gp, ABCB1) and ABCB5 proteins in MDA-MB-231/Luc, hBCSCs and canine MDCK-MDR1 cells. Homogenates (20 pg protein) from MDA-MB-231/Luc, hBCSCs and canine MDCK-MDR1 cells were separated on a polyacrylamide gel and electrotransferred to a PVDF membrane. MDR1 and ABCB5 were detected with specific antibodies on the membrane by Western blotting. For FIGS. 6B-D, hBCSC cells were pre-incubated for 30 min±10 μM CsA (n=4) or 10 μM PSC-833 (n=3). Cells were then incubated for 2 h with vehicle (Control, DMSO), 4 μM Docetaxel or 2 μM TH1902. Media was then replaced by fresh media containing or lacking 10 μM CsA or PSC-833. Cells were incubated for 22 h before staining with the FxCycle™ PI/RNase Staining Solution followed by analysis via flow cytometry. Values shown represent the mean±SEM from at least 3 different experiments and statistical analysis was performed using Bonferroni's multiple comparisons test (** p<0.01, *** p<0.001). FIG. 6B: Representative flow cytometry acquisition plots for each condition. FIG. 6C: graph representing the relative frequency of hBCSCs in G2/M phase following treatment with Docetaxel. FIG. 6D: graph representing the relative frequency of hBCSCs in G2/M phase following treatment with TH1902.

FIG. 7A is a graph showing tumor progression over time in mice following implantation of hBCSCs (with or without Matrigel). 10³ cancer stem-like cells from human TNBC were injected subcutaneously in immunodeficient-mice and tumor volumes were monitored for up to 23 to 28 days.

FIG. 7B is a graph showing tumor progression over time in mice following implantation of human ovarian cancer stem cell (hOvCSCs) with Matrigel™. 10³ cancer stem-like cells from human ovarian cancer were injected subcutaneously in immunodeficient-mice and tumor volumes were monitored for up to 23 to 28 days.

FIG. 7C is a graph showing tumor progression over time in mice following implantation of human pancreatic cancer stem cell (hPCSCs) with Matrigel™. 10³ cancer stem-like cells from human pancreatic cancer were injected subcutaneously in immunodeficient-mice and tumor volumes were monitored for up to 23 to 28 days.

FIG. 7D is a Western Blot showing the expression of multidrug proteins ABCB5 and Pgp, and Sortilin (intracellular), by human breast CSCs, human pancreatic CSCs and human ovarian CSCs.

FIG. 7E is a Western Blot showing the expression of Pgp and Sortilin by human pancreatic CSCs and ovarian CSCs. Expression of β-actin was used as a control.

FIGS. 8A-C show the growth of hBCSC xenografts within mice following administration of Docetaxel or TH1902. FIG. 8A: At day 2 following subcutaneous implantation of hBCSC xenografts within immunodeficient mice, animals began receiving weekly administration of vehicle or vehicle containing docetaxel at 3.75 mg/kg (corresponding to ¼ of the maximum tolerated dose (MTD)) (Docetaxel ¼), docetaxel at 15 mg/kg (corresponding to the MTD) (Docetaxel), TH1902 at 8.75 mg/kg (eq. to ¼ of docetaxel MTD) (TH1902 ¼) and TH1902 at 35 mg/kg (eq. to docetaxel MTD) (TH1902). The tumor size was measured manually at regular intervals in order to monitor the effects of test article administration on tumor growth. Symbols represent means±SEM, n=6. Note that there is virtual overlap between the 15 mg/kg docetaxel curve and the 8.75 mg/kg TH1902 curve. The symbols displayed below the abscissa denote the days on which treatments occurred. Note also that docetaxel (15 mg/kg) was only administered three times as that combined amount matches the MTD for docetaxel in mice. FIG. 8B: Tumor volume comparison following treatment with docetaxel or TH1902. Statistical comparison between the different treatments of hBCSCs was performed by calculating the change in tumor sizes between the first day of treatment and the last day of the experiment. The sizes were compared by one-way ANOVA and the mean values for tumor sizes associated with each treatment were compared to the tumor sizes for vehicle-treated animals using Dunnett's multiple comparison test with p<0.05 as the pre-set level of significance. The treatments listed along the abscissa identify the treatment along with the concentration used (in mg/kg) listed in brackets. ns: not significative, *P<0.05, **** P<0.0001, n=6. FIG. 8C is a graph showing changes in mice weight over time in the study described in FIG. 8A. Body weights of the mice were routinely recorded and considered as a rough estimate of morbidity. The data is shown here as a percentage of the animal's body weight at the beginning of treatment. Symbols represent means±SEM, n=6 mice/group. The symbols displayed below the abscissa indicate the days on which treatments occurred.

FIGS. 9A-C show the effect of docetaxel and increasing TH1902 doses on the growth of hBCSC xenografts in mice. FIG. 9A: At day 3 following subcutaneous implantation of hBCSC xenografts within immunodeficient mice (n=6 mice per group), animals began receiving weekly administration of vehicle or vehicle containing docetaxel at 15 mg/kg (corresponding to the MTD) (Docetaxel), TH1902 at 35 mg/kg (eq. to docetaxel MTD) (TH1902 1 eq.), TH1902 at 43.75 mg/kg (eq. to 1.25 docetaxel MTD) (TH1902 1.25 eq.) and TH1902 at 52.5 mg/kg (eq. to 1.5 docetaxel MTD) (TH1902 1.5 eq.). The tumor size was measured manually at regular intervals in order to monitor the effects of test article administration on tumor growth. Symbols represent means±SEM. The symbols displayed below the abscissa denote the days on which treatments occurred. FIG. 9B: Tumor volume comparison following treatment with docetaxel or TH1902. Statistical comparison between the different treatments of hBCSCs was performed by calculating the change in tumor sizes between the first day of treatment and the last day of the experiment. The sizes were compared by one-way ANOVA and the mean values for tumor sizes associated with each treatment were compared to the tumor sizes for vehicle-treated animals using Dunnett's multiple comparison test with p<0.05 as the pre-set level of significance. The treatments listed along the abscissa identify the treatment along with the concentration used (in mg/kg) listed in brackets. One-way ANOVA turkey's multiple comparisons test analysis. ns: not significative, *P<0.05, **P<0.01, **** P<0.0001, n=6 per group except for TH1902 52.5 mg/kg n=5. FIG. 9C is a graph showing changes in mice weight over time in the study described in FIG. 9A. Body weights of the mice were routinely recorded and considered as a rough estimate of morbidity. The data is shown here as a percentage of the animal's body weight at the beginning of treatment. Symbols represent means±SEM, n=6 mice/group. The symbols displayed below the abscissa indicate the days on which treatments occurred.

FIGS. 10A-C show the effect of docetaxel and increasing TH1902 doses on the growth of hOvCSC xenografts in mice. FIG. 10A: At day 3 following subcutaneous implantation of hOvCSC xenografts within nude mice (n=5 to 6 mice per group), animals began receiving weekly administration of vehicle or vehicle containing docetaxel at 15 mg/kg (corresponding to the MTD) (Docetaxel), TH1902 at 35 mg/kg (eq. to docetaxel MTD) (TH1902 1 eq.), TH1902 at 43.75 mg/kg (eq. to 1.25 docetaxel MTD) (TH1902 1.25 eq.) and TH1902 at 52.5 mg/kg (eq. to 1.5 docetaxel MTD) (TH1902 1.5 eq.). The tumor size was measured manually at regular intervals in order to monitor the effects of test article administration on tumor growth. Symbols represent means±SEM. The symbols displayed below the abscissa denote the days on which treatments occurred. FIG. 10B: Tumor volume comparison following treatment with docetaxel or TH1902. Statistical comparison between the different treatments of hOvCSC was performed by calculating the change in tumor sizes between the first day of treatment and the last day of the experiment. The sizes were compared by one-way ANOVA and the mean values for tumor sizes associated with each treatment were compared to the tumor sizes for vehicle-treated animals using Dunnett's multiple comparison test with p<0.05 as the pre-set level of significance. The treatments listed along the abscissa identify the treatment along with the concentration used (in mg/kg) listed in brackets. One-way ANOVA turkey's multiple comparisons test analysis. ***P<0.001, **** P<0.0001. FIG. 10C is a graph showing changes in mice weight over time in the study described in FIG. 10A. Body weights of the mice were routinely recorded and considered as a rough estimate of morbidity. The data is shown here as a percentage of the animal's body weight at the beginning of treatment. Symbols represent means±SEM, n=5 or 6 mice/group. The symbols displayed below the abscissa indicate the days on which treatments occurred.

FIGS. 11A and 11B show the effect of docetaxel, paclitaxel, TH1902 or carboplatin used alone or in combination on the growth of hOvCSC xenografts in mice. Groups of mice bearing hOCSC xenografts were administered with docetaxel, TH1902 or paclitaxel intravenously, with and without intraperitoneal injection of carboplatin. FIG. 11A is a graph showing tumor growth over time. The circles shown beneath the abscissa indicate the days on which treatments were administered. Symbols represent means±SEM, n=6 for all groups. FIG. 11B is a graph showing a comparison of tumor sizes following treatments. Tumor sizes on day 18 (final day of experiment) were measured for all animals and these sizes were compared between the groups receiving different treatments. Statistical comparison was performed using a one-way ANOVA followed by Tukey's multiple comparisons test with statistical significance pre-set at p<0.05. * denotes p<0.05, ** denotes p<0.01, *** denotes p<0.001 and **** denotes p<0.0001. n=6 for all groups. The box ends represent the median values of the lower and upper halves of the datasets and the whiskers show the minimum and maximum values of each set. The vertical line inside the box marks the median value for that set. Pairwise comparisons that are not marked in the figure were not statistically significant.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

Described herein are products and methods and uses thereof for targeting cancer stem cells (CSCs). CSCs have been associated with cancer relapse, metastasis, multidrug resistance, and radiation resistance through their ability to arrest in the G0 phase, giving rise to new tumors. Solid tumors are composed of heterogeneous cell populations with different phenotypic characteristics at different stages of development, with variable abilities to proliferate. CSCs have been defined as a small subpopulation of cancer cells within cancer microenvironment and niche that establish a reservoir of self-sustaining cells with the exclusive ability to self-renew and to form the heterogeneous lineages of cancer cells that comprise the tumor. High levels of circulating cancer stem-like cells (cCSCs) have been associated with an inferior tumor response rate to chemotherapy and lower overall and progression-free survival in breast cancer patients (Lee, C H et al., BMC Cancer 19, 1167 (2019)). Similarly, high stem cell frequency at diagnosis correlates with lower response to chemotherapy and relapse, and in turn lower survival, in acute myeloid leukemia (AML) patients (van Rhenen et al., Clin Cancer Res. 2005 Sep 15; 11(18):6520-7; Witte K E et al., Pediatr Hematol Oncol. 2011 March; 28(2):91-9). High frequency of CSCs correlates with higher metastatic potential and worse prognosis in melanoma (Civenni et al., Cancer Res. 2011 Apr 15; 71(8):3098-109).

The results presented herein show that Triple-Negative Human Breast Cancer Stem Cells (hBCSCs) express the Sortilin receptor, and that such cells are resistant to treatment with Docetaxel alone due the expression of multidrug resistant proteins such as MDR1 and ABCB5, but are sensitive to Docetaxel conjugated to a peptide compound permitting the Sortilin-mediated uptake of Docetaxel by hBCSCs. The conjugate was also shown to prevent cancer relapse in a mouse model of an aggressive triple-negative breast cancer (TNBC). Since CSCs have been associated with cancer relapse, metastasis, multidrug resistance, and radiation resistance, these results provide evidence that conjugates comprising antitumor agents (e.g., chemotherapeutic agents) conjugated to peptide compounds targeting Sortilin-expressing CSCs may be used for the treatment of poor prognosis cancers refractory to standard antitumor therapies, and may prevent or treat cancer relapse or recurrence.

Accordingly, in an aspect, the present disclosure provides a method for treating a cancer comprising Sortilin-expressing cancer stem cells (CSCs) in a subject, the method comprising administering to the subject an effective amount of a conjugate compound defined herein or a pharmaceutically acceptable salt thereof. The present disclosure also provides the use of a conjugate compound defined herein or a pharmaceutically acceptable salt thereof for treating a cancer comprising Sortilin-expressing CSCs in a subject, or for the manufacture or preparation of a medicament for treating a cancer comprising Sortilin-expressing CSCs in a subject. The present disclosure also provides a conjugate compound defined herein or a pharmaceutically acceptable salt thereof for use in treating a cancer comprising Sortilin-expressing CSCs in a subject.

In another aspect, the present disclosure provides a method for reducing or eliminating Sortilin-expressing CSCs in a cancer patient, the method comprising administering to the subject an effective amount of a conjugate compound defined herein or a pharmaceutically acceptable salt thereof. The present disclosure also provides the use of a conjugate compound defined herein or a pharmaceutically acceptable salt thereof for reducing or eliminating Sortilin-expressing CSCs in a cancer patient, or for the manufacture or preparation of a medicament for reducing or eliminating Sortilin-expressing CSCs in a cancer patient. The present disclosure also provides a conjugate compound defined herein or a pharmaceutically acceptable salt thereof for use in reducing or eliminating Sortilin-expressing CSCs in a cancer patient.

In another aspect, the present disclosure provides a method for preventing or treating a cancer relapse or recurrence associated with the presence of Sortilin-expressing CSCs in a subject, the method comprising administering to the subject an effective amount of a conjugate compound defined herein or a pharmaceutically acceptable salt thereof. The present disclosure also provides the use of a conjugate compound defined herein or a pharmaceutically acceptable salt thereof for preventing or treating a cancer relapse or recurrence associated with the presence of Sortilin-expressing CSCs in a subject, or for the manufacture or preparation of a medicament for preventing or treating a cancer relapse or recurrence associated with the presence of Sortilin-expressing CSCs in a subject. The present disclosure also provides a conjugate compound defined herein or a pharmaceutically acceptable salt thereof for use in preventing or treating a cancer relapse or recurrence associated with the presence of Sortilin-expressing CSCs in a subject. In an embodiment, the conjugate compound or salt thereof prevents cancer relapse or recurrence. In an embodiment, the conjugate compound or salt thereof reduces cancer relapse or recurrence. In an embodiment, the conjugate compound or salt thereof treats cancer relapse or recurrence (i.e., treats a relapsing cancer).

The present disclosure also provides a method for treating a poor prognosis cancer associated with the presence of Sortilin-expressing cancer stem cells (CSCs) in a subject comprising administering to the subject an effective amount of a conjugate compound defined herein or a pharmaceutically acceptable salt thereof. The present disclosure also provides the use of a conjugate compound defined herein or a pharmaceutically acceptable salt thereof for treating a poor prognosis cancer associated with the presence of Sortilin-expressing cancer stem cells (CSCs) in a subject, or for the manufacture or preparation of a medicament for treating a poor prognosis cancer associated with the presence of Sortilin-expressing cancer stem cells (CSCs) in a subject. The present disclosure also provides a conjugate compound defined herein or a pharmaceutically acceptable salt thereof for use in treating a poor prognosis cancer associated with the presence of Sortilin-expressing cancer stem cells (CSCs) in a subject.

In another aspect, the present disclosure also provides a method for treating an unresectable, chemotherapy- and radiotherapy-resistant cancer associated with the presence of Sortilin-expressing cancer stem cells (CSCs) in a subject comprising administering to the subject an effective amount of a conjugate compound defined herein or a pharmaceutically acceptable salt thereof. The present disclosure also provides the use of a conjugate compound defined herein or a pharmaceutically acceptable salt thereof for treating an unresectable, chemotherapy- and radiotherapy-resistant cancer associated with the presence of Sortilin-expressing cancer stem cells (CSCs) in a subject, or for the manufacture or preparation of a medicament for treating an unresectable, chemotherapy- and radiotherapy-resistant cancer associated with the presence of Sortilin-expressing cancer stem cells (CSCs) in a subject. The present disclosure also provides a conjugate compound defined herein or a pharmaceutically acceptable salt thereof for use in treating an unresectable, chemotherapy- and radiotherapy-resistant cancer associated with the presence of Sortilin-expressing cancer stem cells (CSCs) in a subject.

The term “Sortilin” or “Sortilin receptor” as used herein refers to a neuronal type-1 membrane glycoprotein, encoded by the SORT1 gene, belonging to the Vacuolar Protein Sorting 10 protein (Vps10) family of receptors. Sortilin (also known as the neurotensin receptor 3; UniProtKB Accession number Q99523) is expressed or overexpressed in a number of cancers including for example ovarian, breast, colon and prostate cancer. The encoded preproprotein (residues 34-831, residues 1-33 corresponding to the signal peptide) is proteolytically processed after amino acid 77 by furin (or other homologous proteases) to generate the mature receptor with a molecular weight of about 100-110 kDa (residues 78-831). Amino acid residues of sortilin referenced herein correspond to positions in the full-length form (i.e., UniProtKB Accession number Q99523).

The term “cancer stem cells” (CSCs) as used herein refers to a subpopulation of cancer cells, found within solid tumors or hematological cancers, that drive tumor initiation and possess characteristics associated with normal stem cells, specifically the ability of self-renewal and differentiation into multiple tumor cell types. CSCs have been shown to exhibit resistance to chemotherapy (multidrug resistance) and radiotherapy, and are associated with cancer relapse and metastasis. Cancer stem cells encompass cells expressing certain markers. Examples of markers of CSCs in various types of cancers are depicted in Table 1 below (see, e.g., Walcher et al., “Cancer Stem Cells—Origins and Biomarkers: Perspectives for Targeted Personalized Therapies”, Front lmmunol. 2020; 11: 1280; Suster et al., “Presence and role of stem cells in ovarian cancer”, World J Stem Cells. 2019 Jul 26; 11(7): 383-397).

TABLE 1
Example of CSC markers in different types of cancers

| Cancer | |
|---|---|
| type | CSC markers |
| Lung | Cell surface: CD44 (and variants), CD87, CD90, CD133, |
| cancer | CD166, EpCAM |
| | Intracellular: ALDH, Nanog, Oct-3/4 |
| CML | Cell surface: CD25, CD26, CD33, CD36, CD117, CD123, |
| | IL1RAP |
| | Intracellular: JAK/STAT, Wnt/β-catenin, FOXO, Hedgehog/ |
| | Smo/Gli2 |
| Breast | Cell surface: CD24, CD29, CD44 (and variants), CD49f, |
| | CD61, CD70, CD90, CD133, CXCR4, EpCAM, LGR5, |
| | ProC-R |
| | Intracellular: ALDH, BMI-1, Nanog, Notch, Oct-3/4, Sox2, |
| | Wnt/β-catenin |
| Gastric | Cell surface: CD24, CD44 (and variants), CD90, CD133, |
| | CXCR4, EpCAM, LGR5, LINGO2 |
| | Intracellular: ALDH, Letm1, Musashi2, Nanog, Oct-3/4, Sox2 |
| Liver | Cell surface: CD24, CD44, CD90, CD133, EpCAM |
| | Intracellular: AFP, Nanog, Notch, Oct-3/4, Sox2, Wnt/β- |
| | catenin |
| Colo- | Cell surface: CD24, CD44, CD133, CD166, EpCAM, LGR5 |
| rectal | Intracellular: ALDH, Letm1, Nanog, Oct-3/4, Sall4, Sox2 |
| AML | Cell surface: CD33, CD123, CLL-1, TIM3 |
| | Intracellular: ALDH, Nanog, Oct-3/4, Sox2 |
| Ovarian | Cell surface: CD24, CD44, CD117, CD133, EpCAM, ROR1 |
| | Intracellular: ALDH, Nanog, Ssea-4, Oct-4, Sox2, MYC |
| Melanoma | Cell surface: Nestin, CD133 |
| Glioma | Cell surface: Nestin, CD133 |

CSCs are also known to express or overexpress multidrug resistance (MDR) proteins (MRPs). MRPs are members of the C family of a group of proteins named ATP-binding cassette (ABC) transporters that efflux a wide spectrum of anticancer drugs against the concentration gradient using ATP-driven energy. The most common MRPs are ABC subfamily C member 1 (ABCC1/MRP1), ABC subfamily C member 2 (ABCC2/MRP2), ABC subfamily C member 3 (ABCC3/MRP3), ABC subfamily C member 4 (ABCC4/MRP4), ABC subfamily C member 5 (ABCC5/MRP5), ABC subfamily C member 6 (ABCC6/MRP6), ABC subfamily C member 10 (ABCC10/MRP7), ABC subfamily C member 11 (ABCC11/MRP8), ABC subfamily C member 12 (ABCC12/MRP9), ABC subfamily B member 1 (ABCB1, also known as P-glycoprotein (P-gp)), ABC subfamily B member 5 (ABCB5) and ABC subfamily G member 2 (ABCG2).

Thus, in an embodiment, the methods and uses defined herein aimed at inhibiting the growth and/or killing of CSCs expressing one or more of the markers listed in Table 1 and/or one or more MRPs such as one or more of the MRPs listed above. In an embodiment, the CSCs express at least one of the following markers: CD133, CD44, SSEA3/4, ALDH and Oct4. In an embodiment, the CSCs express CD133 and/or CD44. In an embodiment, the CSCs express CD133, CD44, SSEA3/4 and Oct4. In an embodiment, the CSCs express Nestin, Sox2, Nanog, cKit and/or Lin28. In another embodiment, the CSCs express ABCB5. In another embodiment, the CSCs express ABCB5 and P-gp. In a further embodiment, the CSCs express CD133 and ABCB5.

The term “Sortilin-positive cancer stem cells” (or “Sortilin-expressing cancer stem cells”) as used herein refers to a population of CSCs in which at least a fraction (e.g., at least 5, 10, 20, 30, 40, 50, 60, 70, 80 or 90%) of the CSCs express or overexpress the native Sortilin receptor. In an embodiment, the Sortilin-positive CSCs are breast CSCs (e.g., CSCs from invasive ductal carcinoma (IDC), triple negative breast cancer (TNBC)), urogenital CSCs (e.g., CSCs from ovarian cancer, prostate cancer, endometrial cancer, testis cancer, urothelial cancer or cervical cancer), head-and-neck CSCs, pancreatic CSCs, lung CSCs, thyroid CSCs, kidney CSCs (e.g., CSCs from renal cell carcinoma (RCC)), gastrointestinal tract CSCs (e.g., CSCs from colorectal cancer, gastric cancer), neuroendocrine CSCs (CSCs from NETs, such as carcinoid), skin CSCs (CSCs from melanoma), brain CSCs (e.g., glioma CSCs), neuroblastoma CSCs, and leukemia CSCs (e.g., CSCs from B cell Chronic Lymphocytic Leukemia, B-CLL) (see, e.g., Mol Cell Proteomics. 2005 December; 4(12):1920-32, the Human Protein Atlas at http://www.proteinatlas.org). In an embodiment, the Sortilin-positive CSCs are breast CSCs, for example TNBC CSCs (e.g., triple negative IDC CSCs). In another embodiment, the Sortilin-positive CSCs are urogenital CSCs. In a further embodiment, the urogenital CSCs are ovarian CSCs. In another embodiment, the urogenital CSCs are prostate CSCs. In another embodiment, the Sortilin-positive CSCs are lung CSCs. In another embodiment, the Sortilin-positive CSCs are pancreatic CSCs. In another embodiment, the Sortilin-positive CSCs are colorectal CSCs. In another embodiment, the Sortilin-positive CSCs are skin CSCs, for example melanoma CSCs.

Thus, in embodiments, the cancer comprising Sortilin-expressing CSCs is breast cancer (e.g., invasive ductal carcinoma (IDC), triple negative breast cancer (TNBC)), urogenital cancer (e.g., ovarian cancer, prostate cancer, endometrial cancer, testis cancer, urothelial cancer, cervical cancer), head-and-neck cancer, pancreatic cancer, lung cancer, thyroid cancer, kidney cancer (e.g., renal cell carcinoma (RCC)), gastrointestinal tract cancer (e.g., colorectal cancer, gastric cancer), neuroendocrine tumors (NETs, such as carcinoid), skin cancer (melanoma), brain cancer (e.g., glioma), neuroblastoma, and leukemia (e.g., B cell Chronic Lymphocytic Leukemia, B-CLL) (see, e.g., Mol Cell Proteomics. 2005 December; 4(12):1920-32, the Human Protein Atlas at http://www.proteinatlas.org). In an embodiment, the cancer comprising Sortilin-expressing CSCs is breast cancer, for example TNBC (e.g., triple negative IDC). In another embodiment, the cancer comprising Sortilin-expressing CSCs is urogenital cancer. In a further embodiment, the urogenital cancer is ovarian cancer. In another embodiment, the urogenital cancer is prostate cancer. In another embodiment, the cancer comprising Sortilin-expressing CSCs is lung cancer. In another embodiment, the cancer comprising Sortilin-expressing CSCs is pancreatic cancer. In another embodiment, the cancer comprising Sortilin-expressing CSCs is colorectal cancer. In another embodiment, the cancer comprising Sortilin-expressing CSCs is skin cancer, for example melanoma.

In an embodiment, the cancer is a relapsing cancer. The terms “cancer recurrence” and “cancer relapse” may be used interchangeably herein, and refer to the return of cancer after treatment and after a period of time during which the cancer cannot be detected. Stated otherwise, it means reappearance of cancer after a disease-free period.

The term “poor prognosis cancer” as used herein refers to a subtype of a given cancer that is associated with lower survival rate (e.g., 5-year or 10-year survival rate) relative to other subtype(s) of the same cancer. Poor prognosis cancer is generally associated with specific characteristics of the cancer subtype, for example the presence of certain mutations, chromosomal abnormalities, etc., that renders them more resistant to treatment. Poor prognosis is also associated with cancers diagnosed at a later stage (e.g., with distant metastasis). Also, as noted above, high CSC frequency has been shown to correlate with poor response to treatment and lower survival in several cancers. For example, for breast cancer, triple-negative breast cancer (TNBC) is considered a poor prognosis breast cancer as it is associated with a lower 5-year relative survival rate relative to other breast cancer subtypes. Also, high levels of circulating cancer stem-like cells (cCSCs) have been associated with an inferior tumor response rate to chemotherapy and lower overall and progression-free survival in breast cancer patients (Lee, C H et al., BMC Cancer 19, 1167 (2019)). For ovarian cancer, invasive epithelial ovarian cancer and fallopian tube cancer are generally associated with a lower 5-year relative survival rate relative to ovarian stromal tumors and germ cell tumors. The 5-year overall survival rate of pancreatic cancer is very low (about 3%), which is partly because more than half of the patients are diagnosed at an advanced stage. Diagnosis of pancreatic cancer at stage III/IV (with distant metastasis) is associated with very poor prognosis. Similarly, for prostate cancer, diagnosis at stage IV (with distant metastasis) is associated with poor prognosis (5-year relative survival rate of less than 30% compared to at least 80-85% for diagnosis at stages I-III). For lung cancer, small cell lung cancer is associated with particularly poor prognosis, especially when diagnosed at a later stage (e.g., with regional or distant metastasis). Non-small cell lung cancer diagnosed at a later stage (e.g., with distant metastasis) is also associated with poor prognosis. In colorectal cancer, mucinous adenocarcinomas (characterized by the presence of abundant extracellular mucin) have been associated with reduced response to chemotherapy and poor prognosis. Peritoneal involvement and BRAF mutations also constitute poor prognosis markers for colorectal cancer. For kidney cancer, clear cell RCC is associated with worse outcomes (e.g., lower 5-year relative survival rate) than papillary RCC. In skin cancer, thicker tumors, nodal involvement and diagnosis at a later stage (e.g., with regional or distant metastasis) are associated with lower survival in melanoma. Expression of Nestin and CD133 has been associated with poor outcome in melanoma and glioma.

In an embodiment, the poor prognosis cancer is a stage III/IV cancer. In an embodiment, the poor prognosis cancer is a stage III cancer. In an embodiment, the poor prognosis cancer is a stage IV cancer. In another embodiment, the poor prognosis cancer is a cancer with a high number or frequency of CSCs, i.e., a number or frequency of CSCs that is higher than the average number or frequency of CSCs in the same type of cancer (e.g., ovarian cancer, breast cancer). In an embodiment, the number or frequency of CSCs is at least 5%, 10%, 20%, 30%, 40%, 50%, 60%, 70%, 80%, 90%, 100% (2-fold), 200% (3-fold), 300% (4-fold) or 400% (5-fold) than the average number or frequency of CSCs in the same type of cancer.

In an embodiment, the poor prognosis cancer is a cancer having a 5-year relative survival rate of less than 60%. In an embodiment, the poor prognosis cancer is a cancer having a 5-year relative survival rate of less than 50%. In an embodiment, the poor prognosis cancer is a cancer having a 5-year relative survival rate of less than 40%. In an embodiment, the poor prognosis cancer is a cancer having a 5-year relative survival rate of less than 30%. In an embodiment, the poor prognosis cancer is a cancer having a 5-year relative survival rate of less than 20%. In an embodiment, the poor prognosis cancer is a cancer having a 5-year relative survival rate of less than 10%. In an embodiment, the poor prognosis cancer is a cancer having a 5-year relative survival rate of less than 5%.

The conjugates suitable for the methods and uses disclosed herein are conjugates comprising antitumor agents (e.g., chemotherapeutic agents) conjugated to agents capable of binding to Sortilin and being internalized by CSCs so as to deliver the antitumor agent to the CSCs. In an embodiment, the conjugate (or conjugate compound) is an antitumor agent-peptide compound conjugate as described in PCT publications Nos. WO/2017/088058, WO/2018/213928 and WO/2020/037434.

In an embodiment, the conjugate compound is of the formula A-(B)ₙ, wherein
A is a peptide compound having an amino acid sequence having at least 60% sequence identity to one of the sequences of formulas (I)-(XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)NPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid; X₁₆, X₁₇, X₂₀ and X₂₁ are independently chosen from Q, P, Y, I and L; n is is an integer from 1 to 10, for example 1, 2, 3, 4 or 5; when X₉ is present more than once, each of said X₉ is independently chosen from any amino acid; when X₁₉ is present more than once, each of said X₉ is independently chosen from any amino acid, optionally the peptide compound is cyclic, and wherein the peptide compound binds to Sortilin.
B is at least one antitumor agent, wherein B is connected to A either directly or via a linker, optionally at a free amine of said peptide compound, at an N-terminal position of said peptide compound, at a free —SH of said peptide compound, or at a free carboxyl of said peptide compound,
or a pharmaceutically acceptable salt thereof.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “non-standard,” “unnatural” or “unusual” refers to the wide selection of non-natural, rare or synthetic amino acids such as those described by Hunt, S. in Chemistry and Biochemistry of the Amino Acids, Barrett, G.C., ed., Chapman and Hall: New York, 1985. Some examples of non-standard amino acids include non-alpha amino acids and D-amino acids. In an embodiment, the peptide compound comprises only natural amino acids. In another embodiment, the peptide compound comprises one or more non-natural or synthetic amino acids, such as D-amino acids.

The expression “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The expression “pharmaceutically acceptable” means compatible with the treatment of subjects such as animals or humans. Also provided herein is a pharmaceutically acceptable salt of a conjugate compound described herein. The expression “pharmaceutically acceptable salt” means an acid addition salt or basic addition salt which is suitable for or compatible with the treatment of subjects such as animals or humans. The expression “pharmaceutically acceptable acid addition salt” as used herein means any non-toxic organic or inorganic salt of any compound of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluenesulfonic and methanesulfonic acids. Either the mono or di-acid salts can be formed, and such salts may exist in either a hydrated, solvated or substantially anhydrous form. In general, the acid addition salts of the compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of the compounds of the present disclosure, for laboratory use, or for subsequent conversion to a pharmaceutically acceptable acid addition salt. The expression “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where CO₂H is a functional group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds or conjugate compounds of the disclosure, for laboratory use, or for subsequent conversion to a pharmaceutically acceptable acid addition salt.

In an embodiment, the peptide compound comprises or consists of the sequences of any one of formulas (I)-(XIII). In embodiments, the peptide compound comprises 50, 45, 40, 35, 30, 25, or 20 amino acids or less.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (I) or SEQ ID NO: 1, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (II) or SEQ ID NO: 2, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (III) or SEQ ID NO: 3, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IV) or SEQ ID NO: 4, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (V) or SEQ ID NO: 5, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VI) or SEQ ID NO: 6, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VII) or SEQ ID NO: 7, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VIII) or SEQ ID NO: 8, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IX) or SEQ ID NO: 9, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (X) or SEQ ID NO: 10, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XI) or SEQ ID NO: 11, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XII) or SEQ ID NO: 12, wherein the peptide compound binds to Sortilin.

In embodiments, the peptide compound has an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XIII) or SEQ ID NO:13, wherein the peptide compound binds to Sortilin.

In an embodiment, the peptide compound comprises 30, 25 or 20 residues or less and comprises the sequence GVRAKAGVRN(Nle)FKSESY (SEQ ID NO:10). In another embodiment, the peptide compound comprises 30, 25 or 20 residues or less and comprises the sequence GVRAKAGVRN(Nle)FKSESYC (SEQ ID NO:31).

In an embodiment, at least one modifying group is connected to said peptide compound at an N- and/or C-terminal end. In an embodiment, the peptide compound comprises a modifying group at the N-terminal end. In an embodiment, the peptide compound comprises a modifying group at the C-terminal end. Such modifying groups may be useful for protecting the peptide compound from modification or degradation (e.g., protease degradation). In an embodiment, the amino-terminal modifying group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched). In a further embodiment, the amino-terminal modifying group is an acetyl group (CH₃—CO—, Ac) or a succinyl group (CO—CH₂—CH₂—CO—). The carboxy-terminal modifying group may be, e.g., a hydroxylamine group (NHOH) attached to the carboxyl group (—C(=O)—NHOH), or an amine attached to the carboxyl group (—C(=O)—NRR′), the amine being a primary, secondary or tertiary amine, and preferably the amine is an aliphatic amine preferably of one to ten carbons, such as methyl amine, iso-butylamine, iso-valerylamine or cyclohexylamine, an aromatic amine or an arylalkyl amine, such as aniline, napthylamine, benzylamine, cinnamylamine, or phenylethylamine, a preferred amine being —NH₂.

In an embodiment, a succinyl group is connected to the peptide compound. For example, the peptide compound has the sequence of Succinyl-IKLSGGVQAKAGVINMFKSESY, corresponding to SEQ ID NO: 6 and having a succinyl group attached thereto at the N-terminal end.

In an embodiment, an acetyl group is connected to the peptide compound. For example, the peptide compound has the sequence of Acetyl-GVRAKAGVRNMFKSESY (SEQ ID NO: 14). For example, the peptide compound has the sequence of Acetyl-GVRAKAGVRN(Nle)FKSESY (SEQ ID NO: 15). For example, the peptide compound has the sequence of Acetyl-YKSLRRKAPRWDAPLRDPALRQLL (SEQ ID NO:16). For example, the peptide compound has the sequence of Acetyl-YKSLRRKAPRWDAYLRDPALRQLL (SEQ ID NO:17). For example, the peptide compound has the sequence of Acetyl-YKSLRRKAPRWDAYLRDPALRPLL (SEQ ID NO:18).

In an embodiment, the peptide compounds can be modified at the C- and/or N-terminal by the addition of one or more (e.g., 1 to 5 or 1 to 3) amino acid residues in order to obtain or increase preferential binding sites at the peptide terminal end. For example, the amino acid can be cysteine. For example, the amino acid can be lysine. For example, the amino acid can be cysteine added at the C-terminal of a peptide. In one embodiment, the peptide compound is modified by the addition of cysteine at the C-terminal. In a specific embodiment, the peptide compound has the sequence of GVRAKAGVRN(Nle)FKSESYC (SEQ ID NO:31) or Acetyl-GVRAKAGVRN(Nle)FKSESYC (SEQ ID NO:32) corresponding to SEQ ID NO:10 and SEQ ID NO:15, respectively, modified by the addition of a cysteine residue at the C-terminal.

The conjugate compound may comprise, for example, from 1 to 10 or from 1 to 5 (e.g., 1, 2, 3 or 4) molecules of an antitumor agent connected thereto. These molecules of antitumor agent can be the same or different, e.g., 2, 3, 4 or more different antitumor agents could be connected to the peptide compounds. The antitumor agent(s) are connected to the peptide compound via at least one covalent bond, at least one atom or at least one linker. In an embodiment, at least 2 molecules of an antitumor agent are attached to A. In an embodiment, the at least two molecules are molecules of the same antitumor agent, e.g., chemotherapeutic agent.

The antitumor agent may be any compound that has the ability to inhibit the growth and/or kill tumor cells and includes, for example, small molecules, peptides, proteins, oligonucleotides (e.g., siRNA, shRNA), radionuclide agents, antibodies, as well as drug delivery systems including nanoparticles, liposomes, nanotubes, graphene particles loaded with a therapeutic antitumor agent.

In an embodiment, the antitumor agent is a chemotherapeutic agent. The term “chemotherapeutic agent” refers to agents that kill tumor cells and/or inhibit their proliferation/growth. Examples in chemotherapeutic agents include alkylating agents (e.g., Cyclophosphamide, Ifosfamide, Mechlorethamine, Chlorambucil, Melphalan, Dacarbazine, Nitrosoureas, Temozolomide, Carmustine, Lomustine, Streptozocin, Busulfan, Procarbazine), anthracyclines (e.g., Daunorubicin, Doxorubicin, Epirubicin, Idarubicin, Mitoxantrone, Valrubicin), cytoskeletal disruptors (e.g., taxanes such as Paclitaxel, Docetaxel, Abraxane, Taxotere, cabazitaxel), histone deacetylase inhibitors (e.g., Vorinostat, Romidepsin), topoisomerase I inhibitors (e.g., Irinotecan, Topotecan), topoisomerase II inhibitors (e.g., Etoposide, Teniposide, Tafluposide), kinase inhibitors (e.g., Bortezomib, Erlotinib, Gefitinib, Imatinib, Vemurafenib, Vismodegib, Dasatinib, Nilotinib, Osimertinib, Crizotinib, Dabrafenib, Vemurafenib, Trametinib, Ibrutinib), nucleotide analogs and precursor analogs (e.g., Azacitidine, Azathioprine, Capecitabine, Cytarabine, Doxifluridine, Fluorouracil (5-FU), Gemcitabine, Hydroxyurea, Mercaptopurine, Methotrexate, Tioguanine (Thioguanine)), peptide antibiotics (e.g., Bleomycin, Actinomycin), platinum-based agents (e.g., Carboplatin, Cisplatin, Oxaliplatin), retinoids (Tretinoin, Alitretinoin, Bexarotene), mitotic inhibitors such as vinca alkaloids and derivative (e.g., Vinblastine, Vincristine, Vindesine, Vinorelbine), toxins such as Maytansinoids, Auristatins, Calicheamicins, Amatoxin or Amanitin, as well as natural phytochemicals having anti-tumor properties such as curcumin, Alkaloids (e.g., Chlorogenic acid, Theobromine, Theophylline), Anthocyanins (e.g., Cyanidin, Malvidin, Carotenoids (Beta-Carotene, Lutein, Lycopene), Coumestans, Flavan-3-OIs, Flavonoids (e.g., Epicatechin, Hesperidin, Isorhamnetin, Kaempferol, Myricetin, Naringin, Nobiletin, Proanthocyanidins, Quercetin, Rutin, Tangeretin), Hydroxycinnamic Acids (e.g., Chicoric acid, Coumarin, Ferulic acid, Scopoletin), Isoflavones (e.g., Daidzein, Genistein), Lignans (e.g., Silymarin), Monoterpenes (e.g., Geraniol, Limonene), Organosulfides (e.g., Allicin, Glutathione, Indole-3-Carbinol, Isothiocyanates, Sulforaphane), Damnacanthal, Digoxin, Phytic acid, Phenolic Acids (e.g., Capsaicin, Ellagic Acid, Gallic acid, Rosmarinic acid, Tannic Acid), Phytosterols (e.g., Beta-Sitosterol), Saponins, Stylbenes (e.g., Pterostilbene, Resveratrol), Triterpenoids (e.g., Ursolic acid), Xanthophylls (e.g., Astaxanthin, Beta-Cryptoxanthin), and Monophenols (e.g., Hydroxytyrosol).

In another embodiment, the antitumor agent is an antibody or antigen-binding fragment thereof that recognizes an antigen expressed by tumor cells, and more specifically CSCs.

In an embodiment, B is connected to A at a free amine of a lysine residue of said peptide compound, optionally via a linker, or at an N-terminal position of said peptide compound, optionally via a linker.

In an embodiment, B is connected to A via a linker, optionally a cleavable linker.

The term “linker” as used herein means a chemical structure connecting a peptide compound disclosed herein to at least one therapeutic agent. The linker can be connected to the peptide compound at different functional groups on the peptide compound. For example, the linker can be connected to the peptide compound at the primary amines (amines (—NH₂): this group exists at the N-terminus of each polypeptide chain (called the alpha-amine) and in the side chain of lysine (Lys, K) residues (called the epsilon-amine). For example, the linker can be connected to the peptide compound at the carboxyls (—COOH): this group exists at the C-terminus of each polypeptide chain and in the side chains of aspartic acid (Asp, D) and glutamic acid (Glu, E). For example, the linker can be connected to the peptide compound at the Sulfhydryls (—SH): This group exists in the side chain of cysteine (Cys, C). Often, as part of a protein's secondary or tertiary structure, cysteines are joined together between their side chains via disulfide bonds (—S—S—). These must be reduced to sulfhydryls to make them available for crosslinking by most types of reactive groups. For example, the linker can be connected to the peptide compound at the Carbonyls (—CHO): Ketone or aldehyde groups can be created in glycoproteins by oxidizing the polysaccharide post-translational modifications (glycosylation) with sodium meta-periodate.

The following table summarizes the reactivity class and the chemical group of some of the principal linkers for standard chemical conjugation:

| Reactivity class | Chemical group |
|---|---|
| Carboxyl-to-amine reactive groups | Carbodiimide (e.g., EDC) |
| Amine-reactive groups | NHS ester |
| | Imidoester |
| | Pentafluorophenyl ester |
| | Hydroxymethyl phosphine |
| Sulfhydryl-reactive groups | Maleimide |
| | Haloacetyl (Bromo- or lodo-) |
| | Pyridyldisulfide |
| | Thiosulfonate |
| | Vinylsulfone |
| Aldehyde-reactive groups | Hydrazide |
| i.e., oxidized sugars (carbonyls) | Alkoxyamine |
| Photoreactive groups | Diazirine |
| | Aryl Azide |

For example, homobifunctional and heterobifunctional crosslinkers can be used. For example, Disuccinimidyl suberate (DSS) is a homobifunctional crosslinker that has identical amine-reactive NHS-ester groups at either end of a short spacer arm. For example, Sulfosuccinimidyl 4-(N-maleimidomethyl)cyclohexane-1-carboxylate (Sulfo-SMCC) is a heterobifunctional crosslinker that has an amine-reactive sulfo-NHS-ester group at one end and a sulfhydryl reactive maleimide group at the opposite end of a cyclohexane spacer arm. This allows for sequential, two-step conjugation procedures. Among the commercially available homobifunctional cross-linkers are: BSOCOES (Bis(2-[Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); and BASED (Bis(β-[4-azidosalicylamido]-ethyl)disulfide iodinatable).

The peptide compounds may be conjugated through a variety of linkers, e.g., sulfhydryl groups, amino groups (amines), or any appropriate reactive group. The linker can be a covalent bond. The linker group may comprise a flexible arm, e.g., 2, 3, 4, 5, 6, 7, 8, 9, 10, 11, 12, 13, 14, or 15 carbon atoms.

Exemplary linkers include, without limitation, pyridinedisulfide, thiosulfonate, vinylsulfonate, isocyanate, imidoester, diazine, hydrazine, thiol, carboxylic acid, multi-peptide linkers, and acetylene. Alternatively other linkers that can be used include BS³ [Bis(sulfosuccinimidyl)suberate] (which is a homobifunctional N-hydroxysuccinimide ester that targets accessible primary amines), NHS/EDC (N-hydroxysuccinimide and 1-ethyl-3-(3-dimethylaminopropyl)carbodiimide (NHS/EDC allows for the conjugation of primary amine groups with carboxyl groups), sulfo-EMCS ([N-ε-maleimidocaproic acid]hydrazide (sulfo-EMCS are heterobifunctional reactive groups that are reactive toward sulfhydryl and amino groups), hydrazide (most proteins contain exposed carbohydrates and hydrazide is a useful reagent for linking carboxyl groups to primary amines).

To form covalent bonds, one can use as a chemically reactive group a wide variety of active carboxyl groups (e.g., esters) where the hydroxyl moiety is physiologically acceptable at the levels required to modify the peptide compound. Particular agents include for example N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA), maleimido hexanoic acid (MHA), and maleimido undecanoic acid (MUA).

Primary amines are the principal targets for NHS esters; NHS esters react with primary amines to form covalent amide bonds. Accessible α-amine groups present on the N-termini of proteins and the ε-amine of lysine react with NHS esters. Thus, conjugated compounds herein disclosed can include a linker having an NHS ester conjugated to an N-terminal amino of a peptide compound, or to an ε-amine of lysine. An amide bond is formed when the NHS ester reacts with primary amines releasing N-hydroxysuccinimide. Succinimide containing reactive groups may be referred to more simply as succinimidyl groups. In some embodiments, the functional group on the peptide compound will be a thiol group and the chemically reactive group will be a maleimido-containing group such as gamma-maleimide-butylamide (GMBA or MPA). Such maleimide-containing groups may be referred to herein as maleido groups.

Amine-to-amine linkers include NHS esters, imidoesters, and others, examples of which are listed below.

Exemplary NHS esters:
DSG (disuccinimidyl glutarate)
DSS (disuccinimidyl suberate)
BS³ (bis[sulfosuccinimidyl] suberate)
TSAT (tris-succinimidyl aminotriacetate)
Variants of bis-succinimide ester-activated compounds including a
polyethylene glycol spacer such as BS(PEG)ₙ where n is 1-20 (e.g.,
BS(PEG)₅ and BS(PEG)₉)
DSP (Dithiobis[succinimidyl propionate])
DTSSP (3,3′-dithiobis[sulfosuccinimidylpropionate])
DST (disuccinimidyl tartarate)
BSOCOES (bis[2-(succinimidooxycarbonyloxy)ethyl]sulfone)
EGS (ethylene glycol bis[succinimidylsuccinate])
sulfo-EGS (ethylene glycol bis[sulfosuccinimidylsuccinate])
Exemplary imidoesters:
DMA (dimethyl adipimidate•2 HCl)
DMP (dimethyl pimelimidate•2 HCl)
DMS (dimethyl suberimidate•2 HCl)
DTBP (dimethyl 3,3′-dithiobispropionimidate•2 HCl)
Other exemplary amine-to-amine linkers:
DFDNB (1,5-difluoro-2,4-dinitrobenzene)
THPP (β-[tris(hydroxymethyl) phosphino] propionic acid (betaine))

The linker may also be a sulfhydryl-to-sulfhydryl linker, such as the maleimides and pyridyldithiols listed below.

| Exemplary maleimides: | Another sulfhydryl linker: |
|---|---|
| BMOE (bis-maleimidoethane) | HBVS (1,6-hexane-bis-vinylsulfone) |

BMB (1,4-bismaleimidobutane)
BMH (bismaleimidohexane)
TMEA (tris[2-maleimidoethyl]amine)
BM(PEG)2 1,8-bis-maleimidodiethyleneglycol)
BM(PEG)ₙ, where n is 1 to 20 (e.g., 2 or 3)
BMDB (1,4 bismaleimidyl-2,3-dihydroxybutane)
DTME (dithio-bismaleimidoethane)
Exemplary pyridyldithiol:
DPDPB (1,4-di-[3′-(2′-pyridyldithio)-propionamido]butane)

The linker may be an amine-to-sulfhydryl linker, which includes NHS ester/maleimide compounds. Examples of these compounds are provided below.

Amine-to-sulfhydryl linkers:

AMAS (N-(α-maleimidoacetoxy)succinimide ester)
BMPS (N-[β-maleimidopropyloxy]succinimide ester)
GMBS (N-[γ-maleimidobutyryloxy]succinimide ester)
sulfo-GMBS (N-[γ-maleimidobutyryloxy]sulfosuccinimide ester)
MBS (m-maleimidobenzoyl-N-hydroxysuccinimide ester)
sulfo-MBS (m-maleimidobenzoyl-N-hydroxysulfosuccinimide ester)
SMCC (succinimidyl 4-[N-maleimidomethyl]cyclohexane-1-carboxylate)
sulfo-SMCC (Sulfosuccinimidyl 4-[N-maleimidomethyl]cyclohexane-1-
carboxylate)
EMCS ([N-ε-maleimidocaproyloxy]succinimide ester)
Sulfo-EMCS ([N-ε-maleimidocaproyloxy]sulfosuccinimide ester)
SMPB (succinimidyl 4-[p-maleimidophenyl]butyrate)
sulfo-SMPB (sulfosuccinimidyl 4-[p-maleimidophenyl]butyrate)
SMPH (succinimidyl-6-[β-maleimidopropionamido]hexanoate)
LC-SMCC (succinimidyl-4-[N-maleimidomethyl]cyclohexane-1-carboxy-
[6-amidocaproate])
sulfo-KMUS (N-[κ-maleimidoundecanoyloxy]sulfosuccinimide ester)
SM(PEG)ₙ (succinimidyl-([N-maleimidopropionamido-
polyethyleneglycol) ester), where n is 1 to 30 (e.g., 2, 4, 6, 8, 12, or 24)
SPDP (N-succinimidyl 3-(2-pyridyldithio)-propionate)
LC-SPDP (succinimidyl 6-(3-[2-pyridyldithio]-propionamido)hexanoate)
sulfo-LC-SPDP (sulfosuccinimidyl 6-(3′-[2-pyridyldithio]-
propionamido)hexanoate)
SMPT (4-succinimidyloxycarbonyl-α-methyl-α-[2-pyridyldithio]toluene)
Sulfo-LC-SMPT (4-sulfosuccinimidyl-6-[α-methyl-α-(2-
pyridyldithio)toluamido]hexanoate)
SIA (N-succinimidyl iodoacetate)
SBAP (succinimidyl 3-[bromoacetamido]propionate)
SIAB (N-succinimidyl[4-iodoacetyl]aminobenzoate)
sulfo-SIAB (N-sulfosuccinimidyl[4-iodoacetyl]aminobenzoate)

The linker can react with an amino group and a non-selective entity. Such linkers include NHS ester/aryl azide and NHS ester/diazirine linkers, examples of which are listed below.

NHS ester/aryl azide linkers:
NHS-ASA (N-hydroxysuccinimidyl-4-azidosalicylic acid)
ANB-NOS (N-5-azido-2-nitrobenzoyloxysuccinimide)
sulfo-HSAB (N-hydroxysulfosuccinimidyl-4-azidobenzoate)
sulfo-NHS-LC-ASA (sulfosuccinimidyl[4-azidosalicylamido]hexanoate)
SANPAH (N-succinimidyl-6-(4′-azido-2′-nitrophenylamino)hexanoate)
sulfo-SANPAH (N-sulfosuccinimidyl-6-(4′-azido-2′-
nitrophenylamino)hexanoate)
sulfo-SFAD (sulfosuccinimidyl-(perfluoroazidobenzamido)-ethyl-1,3′-
dithioproprionate)
sulfo-SAND (sulfosuccinimidyl-2-(m-azido-o-nitrobenzamido)-ethyl-1,3′-
proprionate)
sulfo-SAED (sulfosuccinimidyl 2-[7-amino-4-methylcoumarin-3-
acetamido]ethyl-1,3′dithiopropionate)
NHS ester/diazirine linkers:
SDA (succinimidyl 4,4′-azipentanoate)
LC-SDA (succinimidyl 6-(4,4′-azipentanamido)hexanoate)
SDAD (succinimidyl 2-([4,4′-azipentanamido]ethyl)-1,3′-
dithioproprionate)
sulfo-SDA (sulfosuccinimidyl 4,4′-azipentanoate)
sulfo-LC-SDA (sulfosuccinimidyl 6-(4,4′-azipentanamido)hexanoate)
sulfo-SDAD (sulfosuccinimidyl 2-([4,4′-azipentanamido]ethyl)-1,3′-
dithioproprionate)

Exemplary amine-to-carboxyl linkers include carbodiimide compounds (e.g., DCC (N,N-dicyclohexylcarbodimide) and EDC (1-ethyl-3-[3-dimethylaminopropyl]carbodiimide)). Exemplary sulfhydryl-to-nonselective linkers include pyridyldithiol/aryl azide compounds (e.g., APDP ((N-[4-(p-azidosalicylamido)butyl]-3′-(2′-pyridyldithio)propionamide)). Exemplary sulfhydryl-to-carbohydrate linkers include maleimide/hydrazide compounds (e.g., BMPH (N-[β-maleimidopropionic acid]hydrazide), EMCH ([N-ε-maleimidocaproic acid]hydrazide), MPBH 4-(4-N-maleimidophenyl)butyric acid hydrazide), and KMUH (N-[κ-maleimidoundecanoic acid]hydrazide)) and pyridyldithiol/hydrazide compounds (e.g., PDPH (3-(2-pyridyldithio)propionyl hydrazide)). Exemplary carbohydrate-to-nonselective linkers include hydrazide/aryl azide compounds (e.g., ABH (p-azidobenzoyl hydrazide)). Exemplary hydroxyl-to-sulfhydryl linkers include isocyanate/maleimide compounds (e.g., (N-[p-maleimidophenyl]isocyanate)). Exemplary amine-to-DNA linkers include NHS ester/psoralen compounds (e.g., SPB (succinimidyl-[4-(psoralen-8-yloxy)]-butyrate)).

To generate a branch point of varying complexity in a conjugate peptide compound, the linker can be capable of linking 3-7 entities.

Exemplary tri-functional linkers:

| | | |
|---|---|---|
| TMEA; Tris-(2- maleimidoethyl)amine) 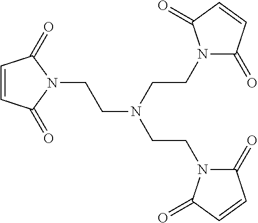 [SMILES: O=C1C=CC(=O)N1CCN(CCN1C(=O)C=CC1=O)CCN1C(=O)C=CC1=O] | THPP 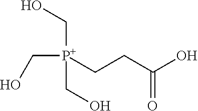 | LC-TSAT (tris-succinimidyl (6- aminocaproyl)aminotriacetate), tris- succinimidyl-1,3,5-benzenetricarboxylate MDSI (maleimido-3,5-disuccinimidyl isophthalate) |
| TSAT; Tris-succinimidyl aminotriacetate 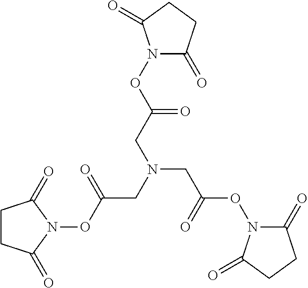 [SMILES: O=C(CN(CC(=O)ON1C(=O)CCC1=O)CC(=O)ON1C(=O)CCC1=O)ON1C(=O)CCC1=O] | | SDMB (succinimidyl-3,5- dimaleimidophenyl benzoate Mal-4 (tetrakis-(3-maleimidopropyl) pentaerythritol, NHS-4 (tetrakis-(N- succinimidylcarboxypropyl)pentaerythritol)) |

TMEA and TSAT reach through their maleimide groups with sulfhydryl groups. The hydroxyl groups and carboxy group of THPP can react with primary or secondary amines. Other useful linkers conform to the formula Y═C═N—Q—A—C(O)—Z, where Q is a homoaromatic or heteroaromatic ring system; A is a single bond or an unsubstituted or substituted divalent C₁₋₃₀ bridging group, Y is O or S; and Z is Cl, Br, I, N₃, N-succinimidyloxy, imidazolyl, 1-benzotriazolyloxy, OAr where Ar is an electron-deficient activating aryl group, or OC(O)R where R is —A—Q—N═C═Y or C₄-20 tertiary-alkyl (see U.S. Pat. No. 4,680,338).

Other useful linkers have the formula

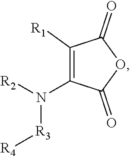

where R₁ is H, C₁₋₆ alkyl, C₂₋₆ alkenyl, C₆₋₁₂ aryl or aralkyl or these coupled with a divalent organic —O—, —S—, or

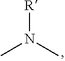

where R′ is C₁₋₆ alkyl, linking moiety; R₂ is H, C₁₋₁₂ alkyl, C₆₋₁₂ aryl, or C₆₋₁₂ aralkyl, R₃ is

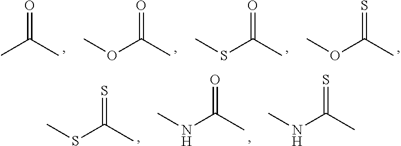

or another chemical structure that is able to delocalize the lone pair electrons of the adjacent nitrogen and R₄ is a pendant reactive group capable of linking R₃ to a peptide compound (see for example U.S. Pat. No. 5,306,809).

The linker may include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly or Cys). Where the linker is a short peptide, it can be a glycine-rich peptide (which tend to be flexible) such as a peptide having the sequence [Gly-Gly-Gly-Gly-Ser]ₙ where n is an integer from 1 to 6, inclusive (see U.S. Pat. No. 7,271,149) or a serine-rich peptide linker (see U.S. Pat. No. 5,525,491). Serine rich peptide linkers include those of the formula [X-X-X-X-Gly]_{y} (SEQ ID NO:19) where up to two of the X are Thr, the remaining X are Ser, and y is an integer greater than 1, for example from 1 to 5, inclusive (e.g., [Ser-Ser-Ser-Ser-Gly]_{y} (SEQ ID NO:20), where y is an integer greater than 1, for example from 1 to 5). Other linkers include rigid linkers (e.g., PAPAP (SEQ ID NO:21) and (PT)ₙP, where n is 2, 3, 4, 5, 6, or 7) and α-helical linkers (e.g., A(EAAAK)ₙA (SEQ ID NO:22), where n is 1, 2, 3, 4, or 5).

The linker can be an aliphatic linker (e.g., with an amide bond to the polypeptide and an ester bond to the therapeutic agent). Where an aliphatic linker is used, it may vary with regard to length (e.g., C₁-C₂₀, C₁-C₁₂, C₁-C₆) and the chemical moieties it includes (e.g., an amino group or carbamate).

Examples of suitable amino acid linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

The linker can also be a branched polypeptide. Exemplary branched peptide linkers are described in U.S. Pat. No. 6,759,509.

The linker can provide a cleavable linkage (e.g., a thioester linkage) or a non-cleavable linkage (e.g., a maleimide linkage). For example, a cytotoxic protein can be bound to a linker that reacts with modified free amines, which are present at lysine residues within the polypeptide and at the amino-terminus of the polypeptide. Thus, linkers useful in the present conjugate compounds can comprise a group that is reactive with a primary amine on the polypeptide or modified polypeptide to which the therapeutic agent moiety is conjugated. More specifically, the linker can be selected from monofluoro cyclooctyne (MFCO), bicyclo[6.1.0]nonyne (BCN), N-succinimidyl-S-acetylthioacetate (SATA), N-succinimidyl-S-acetylthiopropionate (SATP), maleimido and dibenzocyclooctyne ester (a DBCO ester). Useful cyclooctynes, within a given linker, include OCT, ALO, MOFO, DIFO, DIBO, BARAC, DIBAC, and DIMAC.

The linker may comprise a flexible arm, such as for example, a short arm (<2 carbon chain), a medium-size arm (from 2-5 carbon chain), or a long arm (3-6 carbon chain).

Click chemistry can also be used for conjugation on a peptide (DBCO, TCO, tetrazine, azide and alkyne linkers). These families of linkers can be reactive toward amine, carboxyl and sulfhydryl groups. In addition, these linkers can also be biotinylated, pegylated, modified with a fluorescent imaging dye, or phosphoramidited for incorporation onto an oligonucleotide sequence.

In an embodiment, the antitumor agent-peptide compound conjugate is represented by formula (LIII) or (LIV):

(LIII)
(SEQ ID NO: 23)
GVRAK(Z¹)AGVRN(Nle)FK(Z²)SESY;
(LIV)
(SEQ ID NO: 24)
Acetyl-GVRAK(Z¹)AGVRN(NIe)FK(Z²)SESY;

wherein Z¹ and Z² are each independently an antitumor (e.g., chemotherapeutic) agent attached to the lysine (K) residues.

In an embodiment, the conjugate compound is: GVRAK(curcumin)AGVRN(Nle)FK(curcumin)SESY—Formula (XIV) (SEQ ID NO:25) that comprises the peptide compound having SEQ ID NO: 10 wherein each lysine residue has a curcumin molecule connected thereto; or YK(curcumin)SLRRK(curcumin)APRWDAPLRDPALRQLL—Formula (XV) (SEQ ID NO:26) that comprises the peptide compound having SEQ ID NO:11 wherein each lysine residue has a curcumin molecule connected thereto.

In an embodiment, the conjugate compound is Acetyl-GVRAK(curcumin)AGVRN(Nle)FK(curcumin)SESY—Formula (XVI) (SEQ ID NO:27) that comprises the peptide compound having SEQ ID NO:15 wherein each lysine residue has a curcumin molecule connected thereto, or Acetyl-YK(curcumin)SLRRK(curcumin)APRWDAPLRDPALRQLL—Formula (XVII) (SEQ ID NO:28) that comprises the peptide compound having SEQ ID NO:16 wherein each lysine residue has a curcumin molecule connected thereto.

In an embodiment, the conjugate compound is GVRAK(docetaxel)AGVRN(Nle)FK(docetaxel)SESY—Formula (XIX) (SEQ ID NO:29) that comprises the peptide compound having SEQ ID NO:10 wherein each lysine residue has a docetaxel molecule connected thereto.

In another embodiment, the conjugate compound is Acetyl-GVRAK(docetaxel)AGVRN(Nle)FK(docetaxel)SESY—Formula (XXIII) (SEQ ID NO:30) that comprises the peptide compound having SEQ ID NO:15 wherein each lysine residue has a docetaxel molecule connected thereto.

In an embodiment, the conjugate compound is GVRAK(doxorubicin)AGVRN(Nle)FK(doxorubicin)SESY—Formula (XXVI) (SEQ ID NO:33) that comprises the peptide compound having SEQ ID NO:10 wherein each lysine residue has a doxorubicin molecule connected thereto.

In another embodiment, the conjugate compound is Acetyl-GVRAK(doxorubicin)AGVRN(Nle)FK(doxorubicin)SESY—Formula (XXVIII) (SEQ ID NO:34) that comprises the peptide compound having SEQ ID NO:15 wherein each lysine residue has a doxorubicin molecule connected thereto.

In an embodiment, the conjugate compound is GVRAKAGVRN(Nle)FKSESYC(aldoxorubicin)—Formula (LI) (SEQ ID NO:35) that comprises the peptide compound having SEQ ID NO:31 wherein cysteine residue has an aldoxorubicin molecule connected thereto, or that comprises the peptide compound having SEQ ID NO:10 wherein a cysteine residue is added to C-terminal of said peptide compound, and wherein the cysteine residue has an aldoxorubicin molecule connected thereto.

In an embodiment, the conjugate compound is Acetyl-GVRAKAGVRN(Nle)FKSESYC(aldoxorubicin)—Formula (LII) (SEQ ID NO:36) that comprises the peptide compound having SEQ ID NO:32 wherein cysteine residue has an aldoxorubicin molecule connected thereto, or that comprises the peptide compound having SEQ ID NO:15 wherein a cysteine residue is added to C-terminal of said peptide compound, and wherein the cysteine residue has an aldoxorubicin molecule connected thereto.

In an embodiment, the conjugate is administered in the form of a prodrug. The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, the available OH and/or NH₂ in the conjugates of the disclosure may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

Covalent modifications of the conjugate are included within the scope of this disclosure. Covalent modifications include reacting targeted amino acid residues of the conjugate with an organic derivatizing agent that is capable of reacting with selected side chains or the N- or C-terminal residues of the conjugate. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)). Other types of covalent modification of the conjugate included within the scope of this disclosure include linking the conjugate to proteins (e.g., albumin) or to nonproteinaceous polymers, e.g., polyethylene glycol (PEG), polypropylene glycol, or polyoxyalkylenes, which may for example increase the in vivo half-life of the conjugate.

In an embodiment, the conjugate compound or pharmaceutically acceptable salt thereof disclosed herein is formulated into a pharmaceutical composition. In an embodiment, the pharmaceutical composition further comprises a pharmaceutically acceptable carrier or excipient. Such compositions may be prepared in a manner well known in the pharmaceutical art by mixing the conjugate compound having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the conjugate compound by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the conjugate compound or salt thereof by the intravenous or subcutaneous route. In an embodiment, the carriers/excipients are adapted for administration of the conjugate compound by the intravenous route. In another embodiment, the carriers/excipients are adapted for administration of the conjugate compound or salt thereof by the subcutaneous route. In another embodiment, the carriers/excipients are adapted for administration of the conjugate compound or salt thereof by the oral route.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, the composition may include excipients such as one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipients for injectable formulations include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness.

The exact amount/dosage of conjugate to be administered will vary according to factors such as the specific cancer cell involved, and the specific cancer disease; the degree of or involvement or the severity of the cancer disease; the size, age, and general health of the cancer patient; the response of the individual patient; the particular compound administered; the bioavailability characteristics of the preparation administered; the dose regimen selected; whether the conjugate is administered alone or in combination with other agents; pharmacodynamic characteristics of the conjugate and their mode and route of administration; and other relevant characteristics that the physician or as one skilled in the art, will readily determine by the use of known techniques and by observing results obtained under analogous circumstances. The conjugate/composition is suitably administered to the patient at one time or over a series of treatments. Preferably, it is desirable to determine the dose-response curve in vitro, and then in useful animal models prior to testing in humans. The present disclosure provides dosages for the conjugates and compositions comprising same. For example, depending on the type and severity of the disease, about 1 μg/kg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/ 25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The conjugate compound or salt thereof or composition comprising same described herein may be used in combination with one or more additional active agents or therapies (radiotherapy, surgery, vaccines, etc.) for the treatment the targeted disease/condition or for the management of one or more symptoms of the targeted disease/condition (e.g., pain killers, anti-nausea agents, etc.). In an embodiment, the conjugate compound described herein is used in combination with one or more chemotherapeutic agents, immunotherapies, checkpoint inhibitors, cell-based therapies, etc. Examples of chemotherapeutic agents suitable for use in combination with the conjugate described herein include, but are not limited to, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof); geldanamycin or a derivative thereof (such as 17-AAG), and other cancer therapeutic agents recognized in the art. In some embodiments, chemotherapeutic agents for use in combination with the conjugate described herein comprise one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, derivatives thereof, and the like. In an embodiment, the conjugate compound or composition comprising same described herein is used in combination with an EGFR or tyrosine kinase targeting agent, for example an EGFR inhibitor (RTK inhibitor). The conjugate compound or salt thereof or composition comprising same described herein may also be used in combination with one or more therapeutic antibodies or antibody fragments, e.g., therapeutic antibodies or antibody fragments used for the treatment of tumors. Examples of antibodies used for the treatment of cancers include antibodies targeting CD52 (e.g., Alemtuzumab), VEGF/VEGFR (e.g., Bevacizumab, Ramucirumab), EGFR (e.g., Cetuximab, Necitumumab, Panitumumab), CD38 (e.g., Daratumumab, Isatuximab), RANKL (e.g., Denosumab), GD2 (e.g., Dinutuximab, Naxitamab-gqgk), SLAMF7 (e.g., Elotuzumab), HER2 (e.g., Margetuximab-cmkb, Pertuzumab), CCR4 (e.g., Mogamulizumab), CD20 (Obinutuzumab, Ofatumumab, Rituximab), BCMA (e.g., Teclistamab), CD19 (e.g., Tafasitamab), CTLA-4 (e.g., Tremelimumab), LAG-3 (e.g., Relatlimab), PD-1 (e.g., Tislelizumab, Penpulimab, Sintilimab, Toripalimab, Retifanlimab, Dostarlimab), PD-L1 (e.g., Durvalumab, Avelumab, Atezolizumab), EpCAM (e.g., Oportuzumab, Edrecolomab), Nectin-4 (e.g., Enfortumab), CD79b (e.g., Polatuzumab).

The combination of active agents and/or compositions comprising same may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent (e.g., the conjugate compound described herein) may be administered to a patient before, concomitantly, before and after, or after a second active agent (e.g., a chemotherapeutic agent or an immunotherapy) is administered. The agents may in an embodiment be combined/formulated in a single composition and thus administered at the same time.

As used herein, the term “subject” or “patient” denotes a mammal, such as a rodent, a feline, a canine, and a primate. Preferably a subject or patient according to the disclosure is a human.

### EXAMPLES

The present disclosure is illustrated in further details by the following non-limiting examples.

### Example 1: Materials and Methods

Reagents. TH19P01 (GVRAKAGVRN(Nle)FKSESY, SEQ ID NO:10) and TH1902 (Acetyl-GVRAK(docetaxel)AGVRN(Nle)FK(docetaxel)SESY, SEQ ID NO:30) were synthesized as previously described (PCT publication No. WO 2017/088058). TH19P01-Alexa Fluor™ 488 was synthesized by modifying the TH19P01 peptide by adding a cysteine to the C-terminal residue (TH19P01-Cterm_{Cys}) to allow attachment of the Alexa Fluor™ 488 tag. TH19P01-Cterm_{Cys} was dissolved in 15% acetonitrile/formic acid (0.1%) at a concentration of 2.5 mg/ml. Alexa Fluor™ 488 C₅-maleimide (Invitrogen, #A10254) dissolved in DMSO at a concentration of 10 mg/ml was added to the TH19P01-Cterm_{Cys} solution (ratio: 1 mg of fluorescent probe per 5 mg of peptide). This ratio contains an excess of peptide in order to fully conjugate the fluorescent probe in solution. The pH of the mixture was adjusted to 5 with 0.1 N NaOH. The reaction was followed to completion by UPLC-MS (about 5-10 minutes at pH 5), then the Alexa Fluor 488™-labeled TH19P01 peptide (TH19P01-Alexa Fluor™ 488) was purified by preparative scale HPLC on an AKTA purifier using 30 RPC resin with A) 0.1% formic acid in water and B) 0.1% formic acid in ACN. Good fractions were tested, pooled and freeze-dried. TH19P01-Alexa 488 is a yellow powder which was stored in the dark at −20° C. until use. Docetaxel was purchased from Tecoland Inc.

Tumor cell lines. Triple-Negative Human Breast Cancer Stem Cells (hBCSCs) were purchased from Celprogen (San Pedro, Cat. #36102-29). Human triple-negative breast cancer (TNBC)-derived MDA-MB-231/Luc (epithelial breast adenocarcinoma) cells were from Cell Biolabs Inc. (San Diego, CA, #AKR-231). Canine renal epithelial MDCK-MDR1 cells were provided by Dr. Amanda Yancy (AstraZeneca Pharmaceuticals, LP, Wilmington DE, USA) (Cancer Chemother Pharmacol (2005) 56: 173-181).

Cell lines and cell culture. Human Breast Cancer Stem Cell Complete Growth (#M36102-29S) and Undifferentiation (#M36102-29US) media with serum (#M36102-295), along with their corresponding Human Breast Cancer Stem Cell Culture Extracellular Expansion (#E36102-29-T75) or Undifferentiation (#U36102-29-T75) Matrix pre-coated T75 flasks, were obtained from Celprogen. DMEM medium (Wisent, #319-005-CL), 100×non-essential amino acids (NEAA; Hyclone™ Laboratories; GE Healthcare Life Sciences, #30238.01) and fetal bovine serum (FBS; Thermo Fisher Scientific, Inc.; #12483-020) were used for culturing the MDA-MB-231/luc cells. High glucose DMEM was from Wisent (St-Bruno QC, #319-020-CL). Human triple-negative human breast cancer stem cells (hBCSCs, Cat. #36102-29, positive for the markers CD133, CD44, SSEA3/4 and Oct4), human ovarian cancer stem cells (hOvCSC, Cat. #36113-40, positive for the markers CD44, CD133, SSEA 3/4, Oct4, Sox2, Nanog, cKit, Nestin and Lin28) and human pancreatic cancer stem cells (hPCSC, Cat. #36115-42, positive for the markers CD133, CD44, SSEA3/4, Oct4 and Nestin) were purchased from Celprogen (Torrance, CA). Cells were grown as adherent monolayers at 37° C. in a humidified atmosphere (5% CO₂). MDA-MB-231/luc cells were cultured in DMEM supplemented with 10% FBS with 1×NEAA. MDCK-MDR1 cells were grown in high glucose DMEM supplemented with 1×NEAA solution and 10% FBS. The hBCSCs, hOvCSC and hPCSCS were grown in their appropriate growth and undifferentiation media along with serum using the corresponding expansion and undifferentiation matrix pre-coated T75 flasks according to provider's instructions (Celprogen).

For experimental use, cells were detached from the culture flask by a 5-10-minute treatment with trypsin (Wisent, #325-042-CL), then were 10-fold diluted and neutralized by addition of complete culture media. Cell counts were assessed manually by hemocytometer (Hausser Scientific, #3200) following trypan blue (0.4%, ThermoFisher Scientific, #15250061) exclusion staining of cells.

Assessment of Intracellular Fluorescence with TH19P01-Alexa Fluor™ 488. hBCSC were grown in 12-well plates in complete media for 24 hours. In some experiments, cells were washed with HBSS (phenol-free) and incubated in HBSS in the presence or absence of 200 nM of Alexa Fluor™ 488-labeled TH19P01 along with (or lacking) excess unlabeled TH19P01 (50 μM), neurotensin (10 μM) (Ambiopharm; North Augusta, SC; #APi1260) or progranulin (1 nM) (Sigma-Aldrich; Oakville, ON; #K110517-L1). In another experiment, siScrambled- or siSORT1-transfected cells were washed with HBSS and incubated in HBSS in the presence or absence of 200 nM Alexa Fluor™ 488-labeled TH19P01. For both types of experiments, after 2 hours of incubation at 37° C., cells were washed with HBSS, trypsinized, washed again and fluorescence assessed in the FL1 channel using a C6 Accuri™ flow cytometer (BD Biosciences, San Jose, CA).

Sortilin Silencing. hBCSCs were transiently transfected for 24 hours with 100 nM of a scrambled siRNA sequence (AllStar Negative Control siRNA, 1027281) or a human siRNA generated against the SORT1 mRNA (Hs_SORT_5 FlexiTube siRNA: SI03115168; Qiagen, Valencia, CA) using Lipofectamine™ 2000 (ThermoFisher Scientific, Burlington, ON).

Inhibition of hBCSC Migration by Test Articles. Cell migration experiments were carried out using the scratch (wound-healing) assay. Cells (2.8×10⁵ cells /well) were plated in 6-well plates for 24 hours and then scratched using a sterile p200 pipette tip. Cells were washed with serum-free medium to remove the detached cells, then treated for 2 hours with serum-free medium containing vehicle (DMSO), 2 μM Docetaxel, or 1 μM TH1902. Cells were rinsed with complete media and incubated for 48 hours in a fresh complete media. Images were acquired, at 0, 24 and 48 hours after scratching, with an inverted microscope.

Assessment of hBCSC Apoptosis by Flow Cytometry. AnnexinV/PI staining was performed using an Apoptosis Detection Kit (BD Pharmingen, Mississauga ON) according to the manufacturer's instructions. Briefly, hBCSCs (1.3×10⁵ cells/well) were seeded for 24 hours in 12-well plates. Cells were treated in serum-deprived medium for 2 hours with vehicle (DMSO), 4 μM Docetaxel or 2 μM TH1902. Cells were washed with complete medium and incubated in fresh complete medium for 22, 48, and 72 hours. Cells were finally harvested, resuspended in a staining solution of 100 μl of 1×binding buffer containing 5 μL of annexinV-FITC and 5 μL of PI. Cells were incubated for 15 minutes at room temperature in the dark before analysis by flow cytometry. The extent of apoptosis was measured, then analyzed using BD Accuri™ C6 software.

Effects of Docetaxel and TH1902 on Tubulin Polymerization. hBCSCs were grown to 80% confluence on pre-coated 18 mm microscope cover slips (Celprogen, #E36102-29-CS18), rinsed with PBS and exposed to serum-free culture medium containing vehicle (DMSO), 4 μM docetaxel or 2 μM TH1902 at 37° C. Following incubation for 2 hours the cells were rinsed with complete culture medium and incubated in fresh complete medium for 48 hours. Cells were then washed with PHEM buffer (60 mM Pipes, 25 mM HEPES, 10 mM EGTA and 2 mM MgCl₂, pH 6.9) and fixed for 15 minutes with 4% paraformaldehyde, then permeabilized with 1% Triton™ X-100 in PHEM buffer for 5 minutes and washed again with PHEM buffer. The cells were blocked in PBS (2.6 mM KCl, 1.4 mM KH₂PO₄, 136.9 mM NaCl and 6.5 mM Na₂HPO₄·7H₂O; pH 7.2) containing 10% normal goat serum and 0.05% Triton™ and then incubated for 1 hour with 1:2000 anti-α-Tubulin primary monoclonal antibody (clone B-5-1-2, Sigma-Aldrich; #T5168) diluted in washing buffer (PBS containing 5% normal goat serum and 0.025% Triton). Cells were washed with washing buffer and incubated for 1 hour with Alexa Fluor™ 488-conjugated goat anti-mouse secondary antibody (1:1000; Invitrogen; #A-11001), washed with diluted blocking buffer, stained with DAPI (2 pg/ml in PBS, Invitrogen; #D1306) for 3 minutes, washed again in washing blocking buffer and mounted onto slides using Prolong™ Gold antifade reagent (Invitrogen, P36934). Cell images were finally digitalized by confocal microscopy (Nikon A1) and analyzed using NIH ImageJ™ Version 1.4.21 software. The excitation and emission wavelengths used for Alexa Fluor™ 488 were 488 nm and 525 nm, respectively; the excitation and emission wavelengths used for DAPI were 404 nm and 450 nm, respectively.

Cell Cycle Analysis Following Treatment of hBCSC by Test Articles. hBCSCs (2.8×10⁵ cells/well) and MDA-MB-231/luc cells (2.3×10⁵ cells/well) were seeded in 6-well plates one day prior to treatment. To test the effects of Docetaxel and TH1902 on cell cycle phases, hBCSCs and MDA-MB-231/luc cells were treated for 2 hours in serum-free media with vehicle (DMSO), 4 μM docetaxel or 2 μM TH1902 (equimolar of docetaxel), rinsed with complete media and incubated in fresh complete media for 22 and 48 hours. For the experiments with the P-gp inhibitors, hBCSCs were first pre-treated for 30 minutes in serum-free media with vehicle (DMSO), or 10 μM cyclosporine A or PSC-833 (P-gp inhibitors). Next, cells were treated for 2 hours with 4 μM docetaxel or 2 μM TH1902 in the continued presence or absence of the P-gp inhibitors before being rinsed and incubated in fresh complete media in the continued presence or absence of the P-gp inhibitors. Cells were then incubated for 22 and 48 hours. In both assays, following the incubation, cells were detached with trypsin, fixed with ice-cold 70% EtOH and kept overnight at 4° C. Cells were washed once with PBS, after which they were stained for 30 minutes at room temperature with FxCycle™ PI/RNase Staining Solution (Thermo Scientific, Waltham, MA; #F10797). Cells were finally subjected to cell cycle analysis using a BD Accuri™ C6 flow cytometer.

Detection of MDR Proteins by Western Blotting. Cells were homogenized in lysis buffer (1% SDS) supplemented with a complete protease inhibitor cocktail from Calbiochem (San Diego, CA). Cells were incubated at room temperature for 30 minutes with vortexing every 5 minutes. Cells were then sonicated on a sonicator (Sonics, model: Vibra Cell™ VC130) at 80% amplitude for 3 cycles of 3 seconds each and centrifuged at 15,000 g for 10 minutes at 4° C. Protein was quantified using a micro BCA protein assay (Thermo Fisher Scientific, #23235). Equal amounts of protein (20 μg) were separated by SDS-polyacrylamide gel electrophoresis (PAGE). Proteins were then electrotransferred to a polyvinylidene fluoride (PVDF) membrane and blocked for 1 hour at room temperature using 5% non-fat dry milk in Tris-buffered saline (150 mM NaCl, 20 mM Tris-HCl, pH 7.5) containing 0.1% Tween™-20 (TBST). Membranes were washed in TBST and incubated overnight with primary antibodies against α-Pgp (1 μg/ml, Thermo Fisher Scientific #MA1-26528), ABCB5 (1:1,000, Novus Biologicals Centennial, CO, #NBP1-77687), sortilin (a murine mAb (1:1,000, BD Biosciences, San Jose CA) or a rabbit polyclonal Ab (1 μg/ml, Abcam, Cambridge MA)) diluted in TBST containing 3% BSA and 0.05% NaN₃. Membranes were washed in TBST and incubated for 1 hour at room temperature with horseradish peroxidase-conjugated anti-mouse or anti-rabbit IgG (1/5000 dilution, Jackson lmmunoresearch West Grove PA) in TBST containing 5% non-fat dry milk. Membranes were washed again in TBST and signals were detected using enhanced chemiluminescence (Amersham Biosciences, Baie d'Urfe, QC).

In vivo Assessment of Docetaxel and TH1902 Anti-Tumor Efficacy. Tumor xenografts were established by subcutaneous inoculation of 10³ hBCSC cells, hOvCSC and hPCSC resuspended in 100 pL of HBSS containing 50% Matrigel™, into the right flank of NCG triple immunodeficient female mice (Charles River, Saint-Constant QC) under light isoflurane anesthesia. Three days following implantation of either hBCSC or hOvCSCS, mice commenced receiving weekly treatment by intravenous bolus injections of vehicle (10% polysorbate-80, 5% dextrose, 0.04% formic acid pH 4.3), docetaxel (15 mg/kg (the MTD for this agent) or 3.75 mg/kg (¼ MTD for this agent), dissolved in 5% dextrose, 3.3% ethanol, 1.7% Tween™-80) or TH1902 (35 mg/kg or 8.75 mg/kg) dissolved in vehicle. Note that the two TH1902 and docetaxel concentrations contain equimolar amounts of docetaxel due to the two docetaxel moieties per TH1902 molecule. Tumor growth was measured using two-dimensional measurements taken with an electronic caliper, and tumor volume was calculated according to the following formula: tumor volume (mm³)=π/6×length×width². Animal weights were measured three times per week with a precision of 10 mg.

Statistical analysis. The individual statistical tests used are described in the figure legends and respective texts. All statistical analysis were performed using Prism software version 8.3.1 (GraphPad, San Diego CA).

### Example 2: Internalization of Fluorescently Labelled TH19P01 by hBCSCs

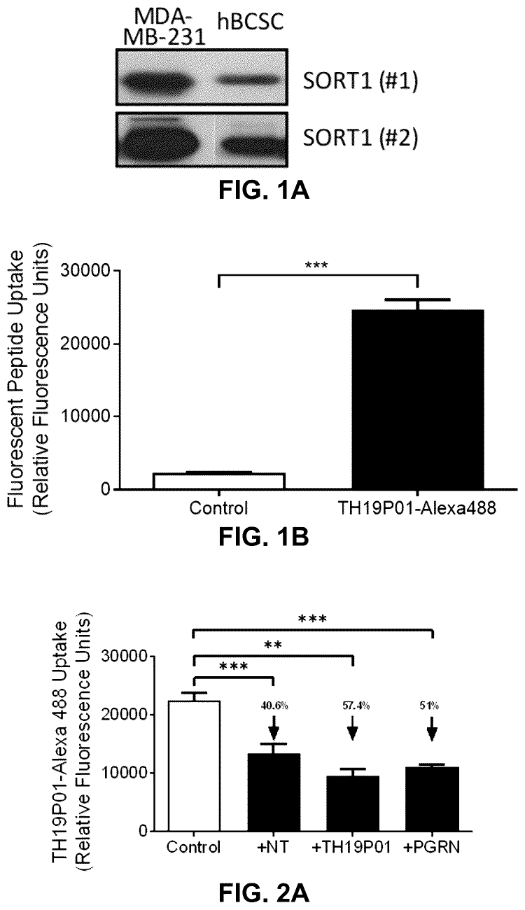

The expression of sortilin in hBCSCs and MDA-MB-231/luc cells was evaluated by Western blot analysis (see FIG. 1A). The results showed that both cell lines contained measurable levels of sortilin. hBCSCs were exposed to either vehicle alone or to vehicle containing 200 nM of the fluorescent peptide TH19P01-Alexa Fluor™ 488 for two hours. The level of fluorescence seen in the vehicle-treated cells is an estimation of the background fluorescence of cells alone measured in the apparatus used. Exposure of cells to TH19P01-Alexa Fluor™ 488 produced a fluorescent signal which is an order of magnitude greater than the background fluorescence and thus easily visible (see FIG. 1B).

### Example 3: Inhibition of TH19P01 Internalization by Sortilin Ligands and Upon Sortilin Silencing

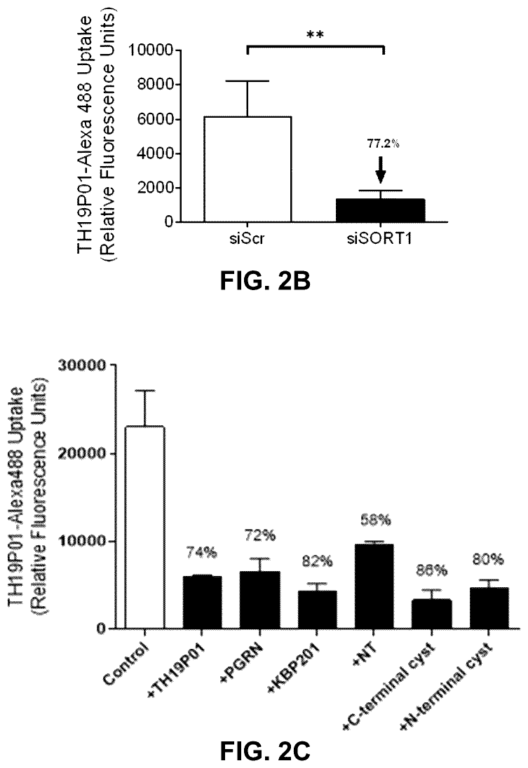

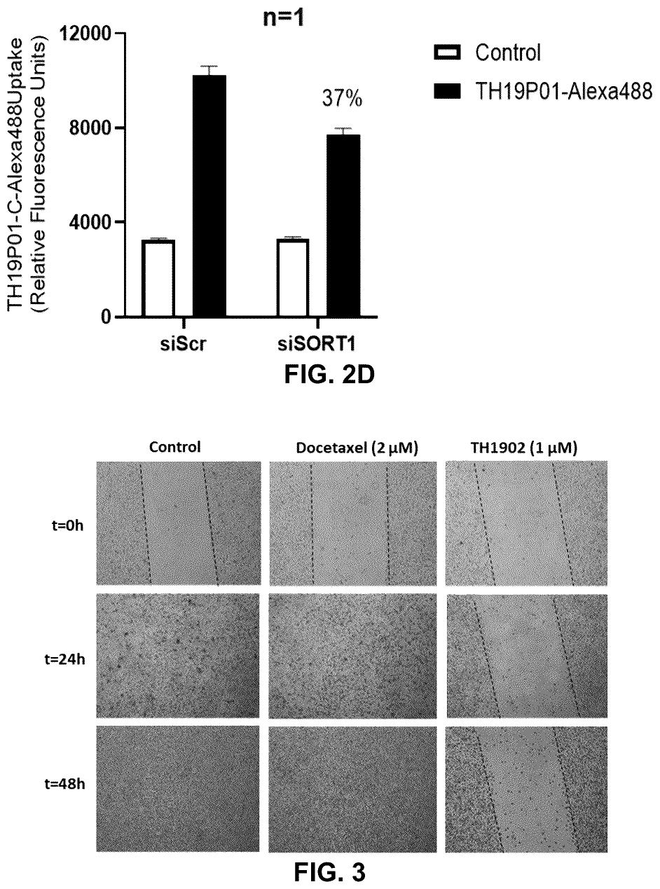

To demonstrate that hBCSC internalization of TH19P01-Alexa Fluor™ 488 is mediated by Sortilin, exposure of the cells to the fluorescent peptide was repeated in the presence or absence of excessive quantities of several known sortilin ligands to determine whether these substances were able to compete with the peptide for internalization. FIG. 2A illustrates the effects of neurotensin (NT), TH19P01 and progranulin (PGRN) on the internalization of fluorescent peptide into the hBCSCs. All three of the competing agents caused the intracellular fluorescence to decrease significantly (p<0.01) by about half, indicating that internalization of the fluorescent peptide had been sharply decreased. Subsequent, confirmation of this finding was provided by pre-incubating hBCSCs in the presence of either scrambled siRNA (siScr) or an siRNA specific for human sortilin (siSORT1). The latter produced a 77% decrease in the amount of intracellular fluorescence following exposure to TH19P01-Alexa Fluor™ 488, again indicating that sortilin is involved in the internalization of this fluorescent peptide in hBCSC. Similar results were obtained in hOvCSCs (FIGS. 2C and 2D), although the decrease in in the amount of intracellular fluorescence following exposure to TH19P01-Alexa Fluor 488 following pre-incubation of the hOvCSCs with a siRNA specific for human sortilin (siSORT1) was less pronounced (37%) (FIG. 2D), suggesting a possible alternative, sortilin-independent internalization mechanism for TH19P01-Alexa Fluor™ 488 in ovarian CSCs.

### Example 4: Inhibition of hBCSC Migration by Docetaxel or TH1902

One standard measurement of cell migration is the scratch assay, where one region of a slide bearing cultured tumor cells is scraped clean of the cells, and the subsequent recolonization of that area by the tumor cells is monitored. As FIG. 3 shows, the initial cell removal provided specific regions cleared of cells. By 24 hours following cell removal, the cleared regions treated with medium containing vehicle, as well as those treated with medium containing 2 μM docetaxel, had been completely recolonized and the denuded regions could no longer be distinguished. In contrast, regions which had been treated with TH1902 remained virtually barren of hBCSC for at least 48 hours. This shows that TH1902 inhibits hBCSC migration.

### Example 5: Induction of hBCSC Apoptosis by Docetaxel or TH1902

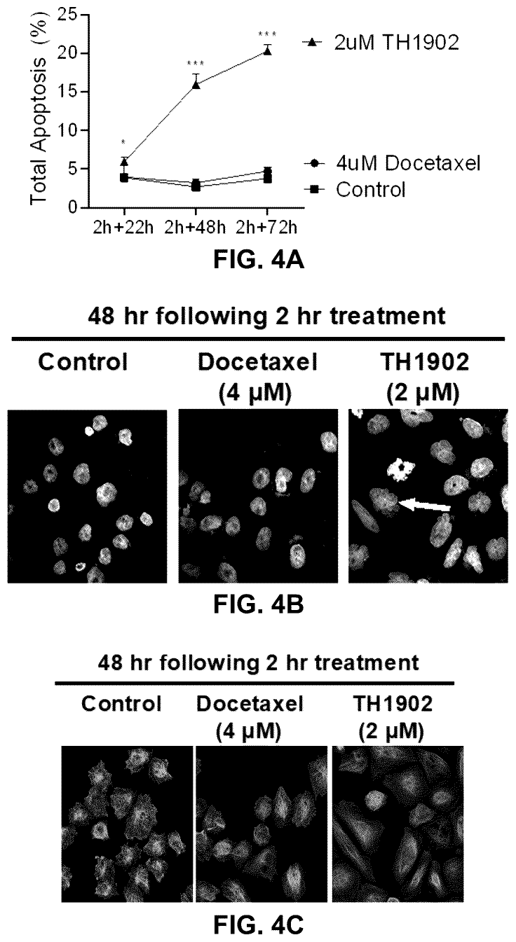

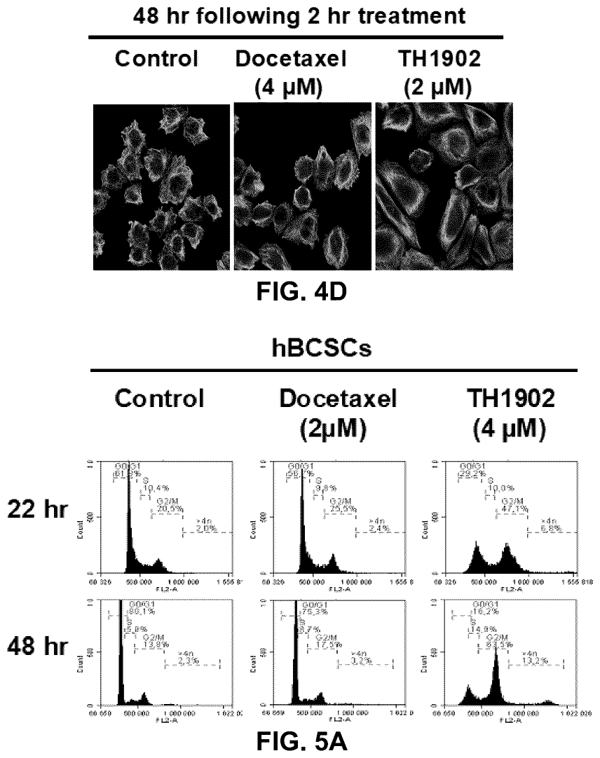

The hBCSCs were exposed to TH1902 or docetaxel to see if they were capable of inducing apoptosis. It is apparent in FIG. 4A that TH1902 produced a large increase in apoptosis in these cells as early as 22 hours post-treatment, which continued to increase over the next 50 hours whereas apoptosis in docetaxel-treated cells is indistinguishable from that seen in control cells. This finding is confirmed by the appearance of fragmented nuclei in many of the cells treated with TH1902 at 48 hours post-treatment (see FIG. 4B), which are not seen in cells treated with vehicle or with docetaxel. Cell morphology was also examined at 48 hours post-treatment. As can be seen in FIG. 4C and FIG. 4C, treatment of hBCSCs with either TH1902 or docetaxel causes a visible morphology change in contrast to vehicle-treated cells. Moreover, the enlargement of cells and the re-organization of the microtubules observed by tubulin labelling is present in some docetaxel-treated but is more evident in TH1902-treated cells. Microtubule perturbation by cytotoxic agents such as docetaxel is known to lead to cell apoptosis.

### Example 6: Comparison of the Induction of G2/M Phase Arrest by Docetaxel and TH1902 in hBCSC vs. MDA-MB-231/luc Cells

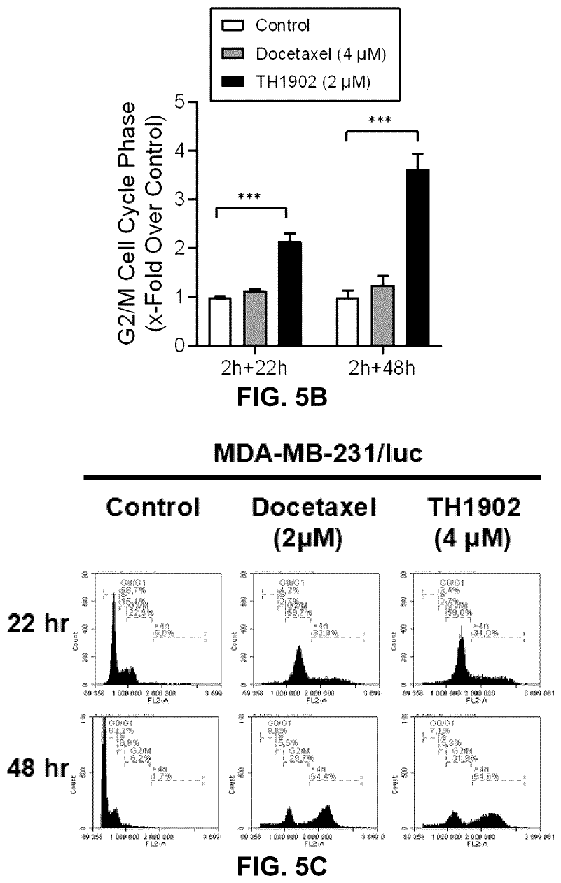

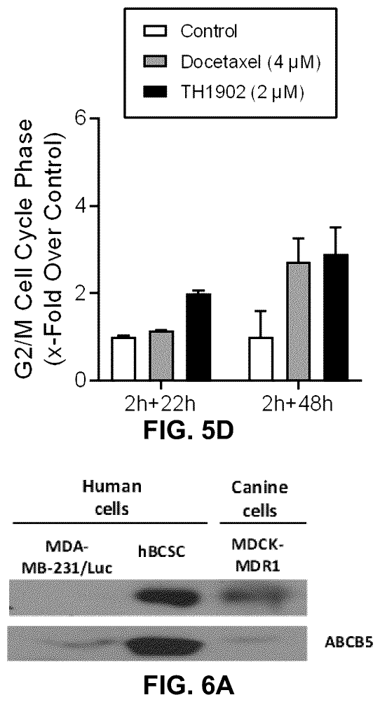

The results reported in FIGS. 5A-B show that TH1902, but not Docetaxel, induces a significant increase in G2/M cell cycle arrest in the hBCSCs. In MDA-MB-231 cells, TH1902 and Docetaxel trigger similar increases in G2/M cell cycle arrest, albeit with differences in the time at which the increased effects become noticeable with some G2/M cell cycle arrest being detected at 22 h in MDA-MB-231 cells treated with TH1902 (but not with Docetaxel) (FIG. 5D, left panel). These results, which are consistent with the results of the apoptosis induction experiments reported in Example 5, provides compelling evidence that TH1902 has the ability to induce cell cycle arrest and apoptosis in hBCSC, which are known to be resistant to treatment with standard cytotoxic agents such as Docetaxel.

### Example 7: Comparison of the Induction of G2/M Phase Arrest by Docetaxel and TH1902 in hBCSC in the Presence of Multidrug-resistance (MDR) Inhibitors

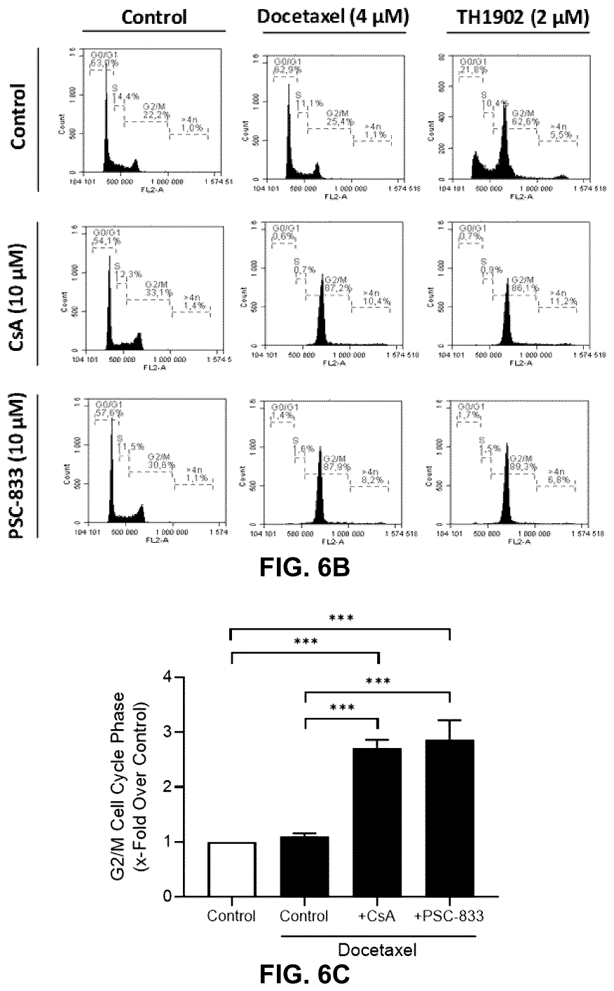

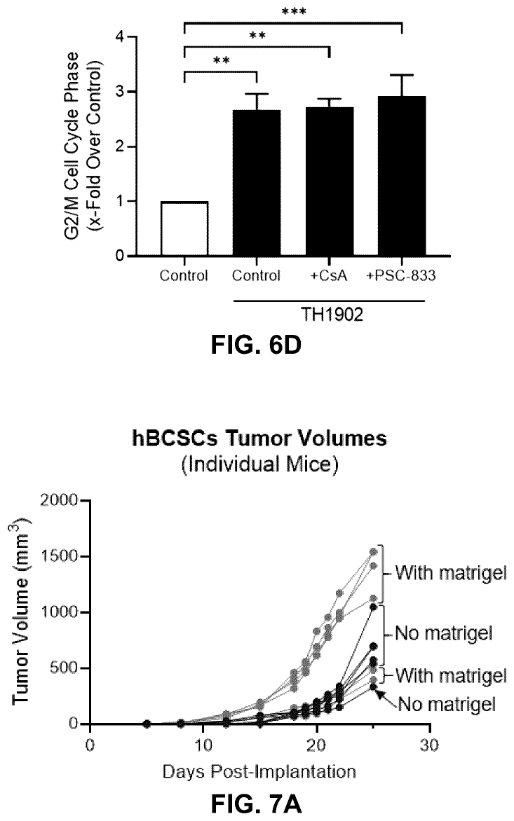

To further investigate the induction of apoptosis by docetaxel and TH1902, these compounds were used to treat hBCSC in the presence of inhibitors of MDR proteins. MDR proteins, which mediate the expulsion of a wide variety of drugs from tumor cells (and are known to limit docetaxel accumulation), are one of the major factors underlying the resistance to chemotherapy. As shown in FIG. 6A, hBCSC express higher levels of MDR1 (P-gp) than the canine cell line MDCK-MDR1, in which the human protein is overexpressed via transfection. In addition, hBCSC were also shown to express high levels of the protein ABCB5 (FIG. 6A), a multi-drug efflux protein related to MDR1. Consequently, these cells provide a good model for examining the involvement of MDR proteins in their resistance to induction of apoptosis by docetaxel.

When the proportions of hBCSC cells in G2/M phase are compared between treatments with docetaxel and with TH1902 (FIG. 6C and FIG. 6D), the first difference noted is that TH1902, but not docetaxel, causes a strong increase in the proportion of cells in G2/M phase (consistent with the results reported in FIGS. 5A-D). The two inhibitors of multi-drug efflux proteins CsA and PCC-831 had no effect on the ability of TH1902 to induce G2/M cell cycle arrest in hBCSCs. In contrast, in the presence of CsA or PCC-831, docetaxel had an effect on G2/M cell cycle arrest that was similar to that seen with TH1902. These results show that the efflux activity of the MDR proteins expressed by hBCSCs confers resistance to docetaxel, but that the cytotoxicity of TH1902 is unaffected by the presence of these proteins, thus providing evidence that TH1902 and other peptide-based conjugates targeting Sortilin may be useful for inhibiting the growth of CSCs expressing or overexpressing MDR proteins.

### Example 8: Administration of TH1902 Prevents Cancer Relapse in the MDA-MB-231/luciferase Xenograft Mouse Model

It was then assessed whether treatment with docetaxel or TH1902 could prevent cancer relapse using the MDA-MB-231/luciferase xenograft mouse model of TNBC. The MDA-MB-231 tumor cell has been reported to have cells with cancer stem cell (CSC)-like properties (Sleeboom et al., Int J Mol Sci. 2018 October; 19(10): 3047; Ghanbari et al., Int. J. Morphol., 34(4):1197-1202, 2016). Mice with MDA-MB-231/luciferase tumor xenografts were treated a day 0, 7, 14 and 21 with either vehicle (control), Docetaxel or TH1902 and tumor growth was assessed up to day 74. It was found that whereas treatment with both Docetaxel and TH1902 led to a reduction in tumor growth during and shortly after administration, only treatment with TH1902 prevented cancer relapse at later time points. No residual tumor could be detected at day 74 in the TH1902-treated group, whereas tumor resurgence was observed in the Docetaxel-treated group. These results provide evidence that TH1902, but not Docetaxel, led to the elimination of all tumor cells including CSCs in the MDA-MB-231 tumor xenografts, and thus prevented cancer relapse due to the persistence of these cells.

### Example 9: Administration of TH1902 Reduces Growth of Breast, Ovarian and Pancreatic CSC Tumors in a Xenograft Mouse Model

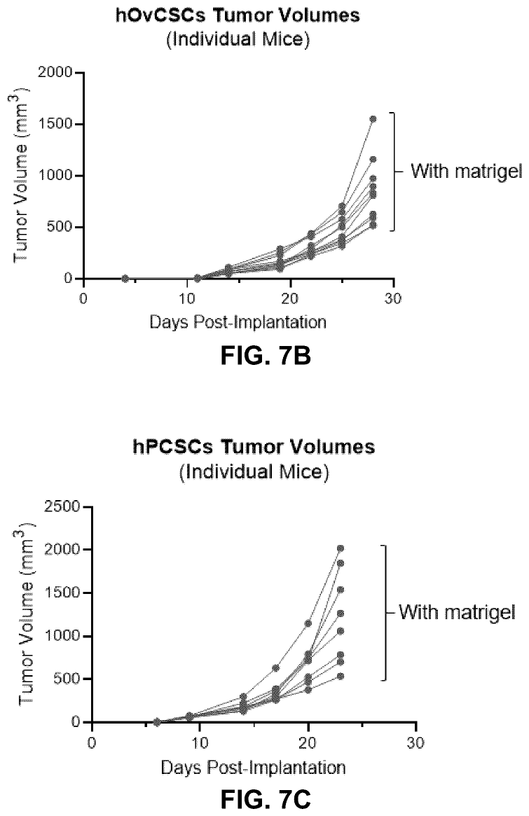

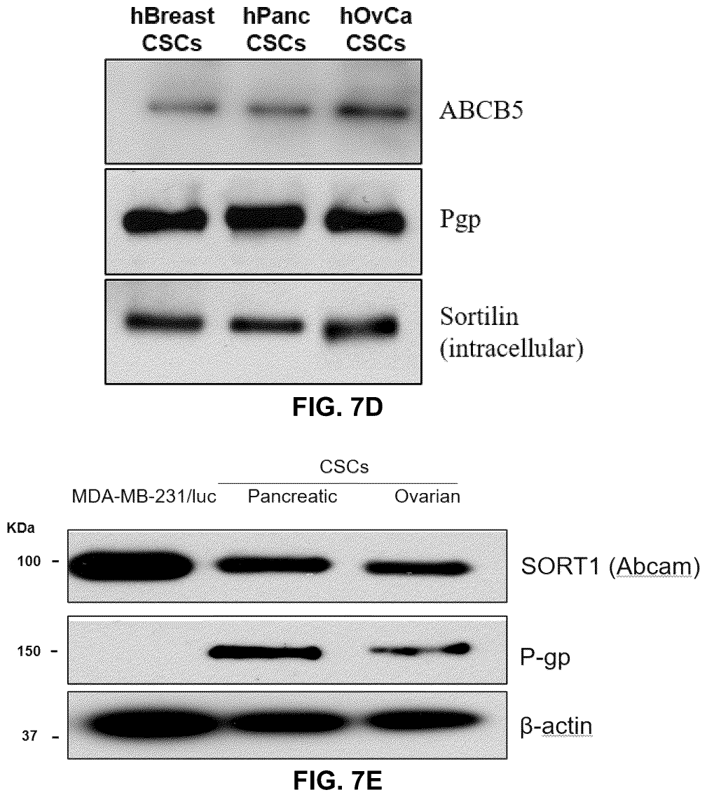

It was next tested whether TH1902 is able to inhibit the growth of human tumors originating from CSCs in vivo. The results presented in FIGS. 7A-C show that subcutaneous injection of cancer stem-like cells from human TNBC (HBCSCs, FIG. 7A), ovarian (HOvCSCs, FIG. 7B) and pancreatic (HPCSCs, FIG. 7C) cancers leads to the growth of tumors in immunodeficient-mice. As shown in FIG. 7D and FIG. 7E, HBCSCs, HOvCSCs and HPCSCs all express the multidrug proteins ABCB5 and P-gp (MDR1), as well as sortilin.

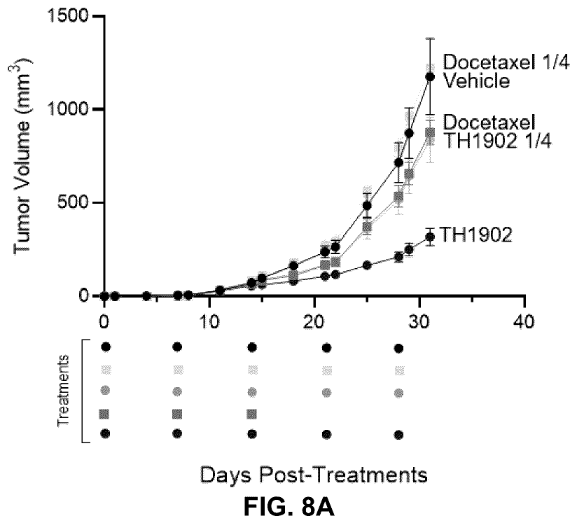

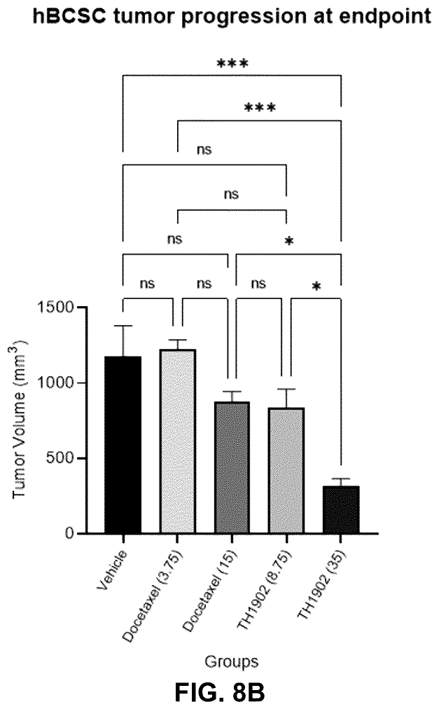

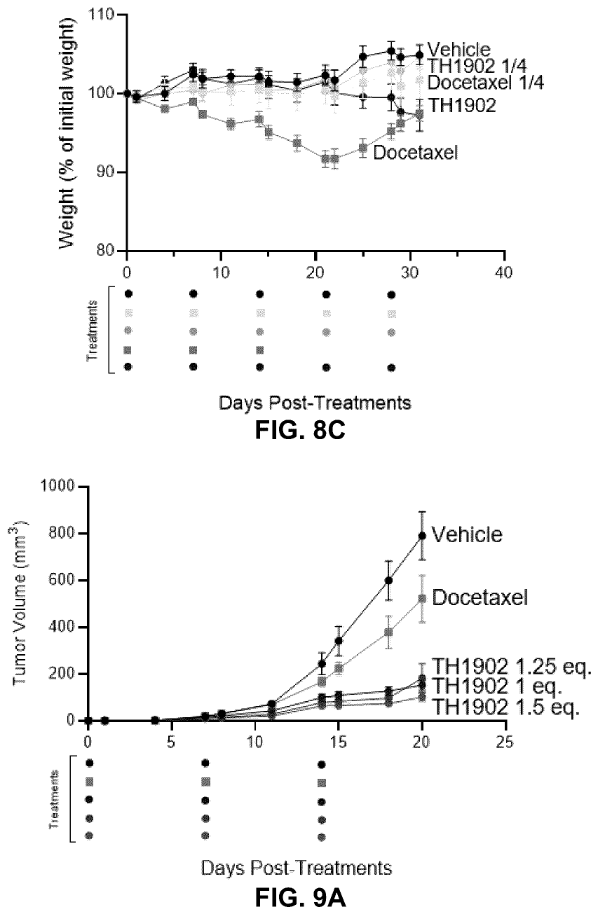

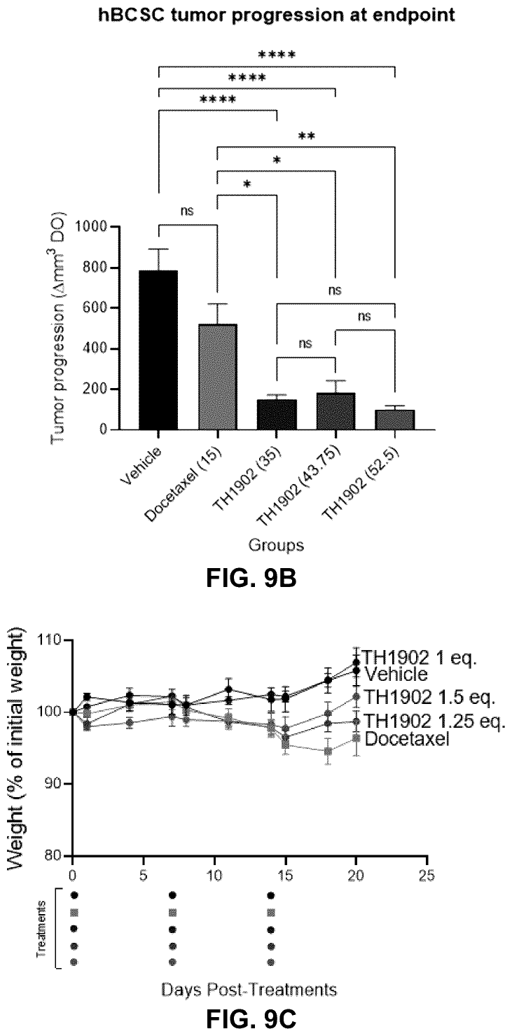

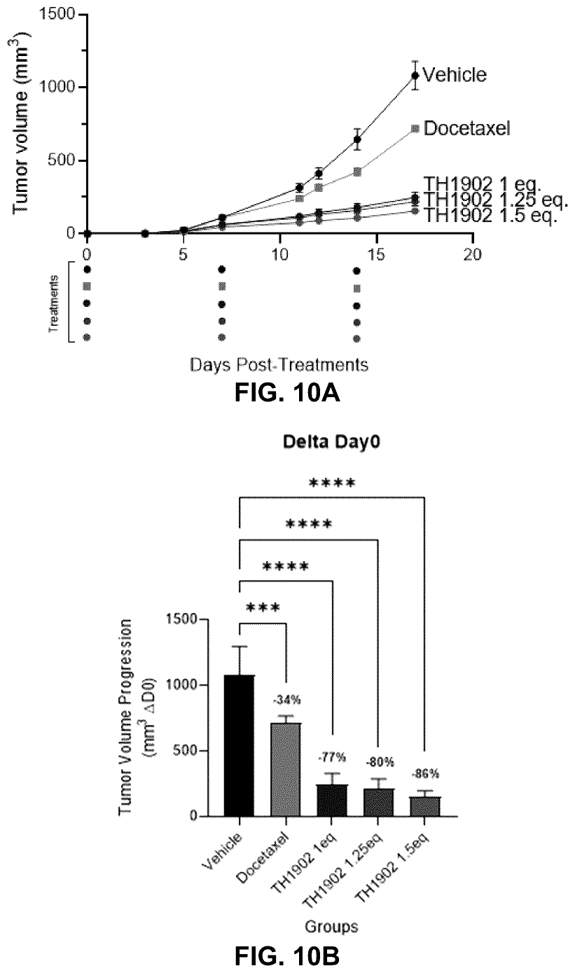

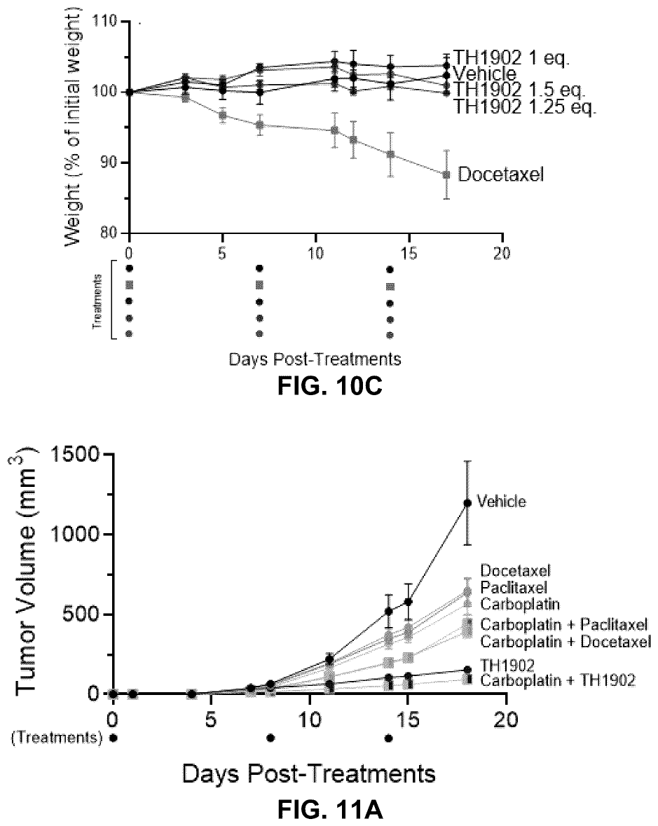

The results presented in FIGS. 8A-B, FIGS. 9A-B, and FIGS. 10A-B show that TH1902 administered at doses equivalent to 1, 1.25 or 1.5 the MTD of docetaxel leads to a significant inhibition of the growth of tumors originating from breast, ovarian and pancreatic CSCs. In contrast, no significant reduction of tumor growth was measured following administration of the MTD of docetaxel (15 mg/kg). Also, administration of TH1902 had no or minimal effect on animals' weight, contrary to administration of docetaxel that was typically associated with a 5-10% weight loss despite the increase in tumor mass in these mice (FIGS. 8C, 9C and 10C).

### Example 10: Inhibition of Ovarian CSC Tumor Growth by Treatment With Combined Chemotherapy

Mice bearing ovarian hOCSC xenografts were treated with vehicle, docetaxel, paclitaxel, TH1902 or carboplatin, and other groups of mice were administered carboplatin in combination with either docetaxel, paclitaxel or TH1902. To minimize the risk of side effects resulting from combining treatments, the dosages used for each drug were reduced to the following: docetaxel 10 mg/kg; TH1902 23 mg/kg (equivalent to the docetaxel dosage); paclitaxel 10 mg/kg; carboplatin 40 mg/kg (unchanged).

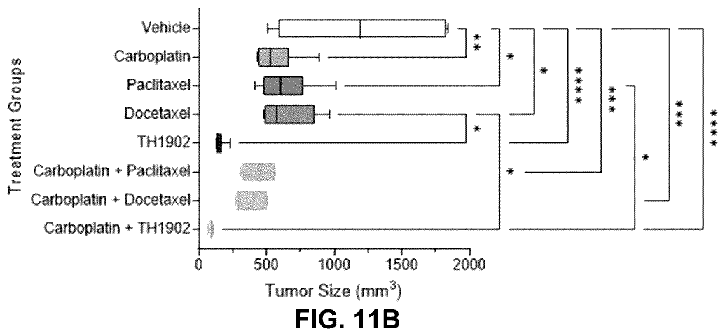

The inhibition of tumor growth by docetaxel was again clearly inferior to that of TH1902 (see FIGS. 11A and 11B). Paclitaxel and carboplatin each showed a level of tumor growth inhibition practically equivalent to that seen with docetaxel. Administration of either taxanes combined with carboplatin produced a small increase in inhibition of tumor growth. The inhibition by TH1902 was much greater than that of either taxanes (with or without carboplatin) and it was slightly increased by combined administration with carboplatin. The extent of tumor growth inhibition by TH1902 alone was so important that the effect of combining administration with carboplatin was difficult to measure.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method for treating a cancer comprising Sortilin-expressing cancer stem cells (CSCs) in a subject comprising administering to the subject an effective amount of a conjugate compound or a pharmaceutically acceptable salt thereof, wherein the conjugate compound has the formula of A-(B)ₙ, wherein
A is a peptide compound of 30 residues or less comprising an amino acid sequence having at least 60% sequence identity to one of the sequences of formulas (I)-(XIII):
(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)_{N}PLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL
wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid,
optionally the peptide compound is cyclic,
B is at least one antitumor agent, wherein B is connected to A directly or via a linker.

2. The method of claim 1, wherein the peptide compound comprises an amino acid sequence having at least 90% sequence identity to one of the sequences of formulas (I)-(XIII).

3. The method of claim 1 or 2, wherein the peptide compound comprises one of the sequences of formulas (I)-(XIII), and further comprises 1 to 3 additional amino acids at its amino- and/or carboxy-terminal end.

4. The method of claim 3, wherein the peptide compound comprises a cysteine residue at its amino- and/or carboxy-terminal end.

5. The method of claim 1, wherein the peptide compound is represented by formula (I) and has an amino acid sequence consisting of the amino acid sequence of SEQ ID NO: 1 or 2.

6. The method of claim 1, wherein the peptide compound is represented by formula (III) and has an amino acid sequence consisting of the amino acid sequence of SEQ ID NO: 3 or 4.

7. The method of claim 1, wherein the peptide compound is represented by formula (V) and has an amino acid sequence consisting of the amino acid sequence of SEQ ID NO: 5.

8. The method of claim 1, wherein the peptide compound is represented by formula (VI) and has an amino acid sequence consisting of the amino acid sequence of SEQ ID NO: 6.

9. The method of claim 1, wherein the peptide compound is represented by formula (VII) and has an amino acid sequence consisting of the amino acid sequence of SEQ ID NO: 7.

10. The method of claim 1, wherein the peptide compound is represented by formula (VIII) and has an amino acid sequence consisting of the amino acid sequence of SEQ ID NO: 8.

11. The method of claim 1, wherein the peptide compound is represented by formula (IX) and has an amino acid sequence consisting of the amino acid sequence of SEQ ID NO: 9.

12. The method of claim 1, wherein the peptide compound is represented by formula (X) and has an amino acid sequence consisting of the amino acid sequence of SEQ ID NO: 10.

13. The method of claim 1, wherein the peptide compound is represented by formula (XI) and has an amino acid sequence consisting of the amino acid sequence of SEQ ID NO: 11.

14. The method of claim 1, wherein the peptide compound is represented by formula (XII) and has an amino acid sequence consisting of the amino acid sequence of SEQ ID NO: 12.

15. The method of claim 1, wherein the peptide compound is represented by formula (XIII) and has an amino acid sequence consisting of the amino acid sequence of SEQ ID NO: 13.

16. The method of any one of claims 1 to 15, wherein the peptide compound comprises at least one modifying group at its amino- and/or carboxy-terminal end.

17. The method of claim 16, wherein the at least one modifying group is acetyl or succinyl.

18. The method of claim 1, wherein the peptide compound is represented by Formula (XXXVIII), (XXXIX), (XL), (XLI) or (XLII):
(XXXVIII)
(SEQ ID NO: 14)
Acetyl-GVRAKAGVRNMFKSESY
(XXXIX)
(SEQ ID NO: 15)
Acetyl-GVRAKAGVRN(Nle)FKSESY
(XL)
(SEQ ID NO: 16)
Acetyl-YKSLRRKAPRWDAPLRDPALRQLL
(XLI)
(SEQ ID NO: 17)
Acetyl-YKSLRRKAPRWDAYLRDPALRQLL
(XLII)
(SEQ ID NO: 18)
Acetyl-YKSLRRKAPRWDAYLRDPALRPLL.

19. The method of any one of claims 1 to 18, wherein B is connected to A at a free amine of said peptide compound, at an N-terminal position of said peptide compound, at a free —SH of said peptide compound, and/or at a free carboxyl of said peptide compound.

20. The method of any one of claims 1 to 19, wherein B is connected to A via a linker.

21. The method of any one of claims 1 to 20, wherein the conjugate is represented by formula (LIII) or (LIV):
(LIII)
(SEQ ID NO: 23)
GVRAK(Z¹)AGVRN(Nle)FK(Z²)SESY;
(LIV)
(SEQ ID NO: 24)
Acetyl-GVRAK(Z¹)AGVRN(Nle)FK(Z²)SESY;
wherein Z¹ and Z² are each independently an antitumor agent attached to the lysine (K) residues.

22. The method of any one of claims 1 to 21, wherein the antitumor agent is a radionuclide or a chemotherapeutic agent.

23. The method of claim 22, wherein the chemotherapeutic agent is a taxane.

24. The method of claim 23, wherein the chemotherapeutic agent is docetaxel.

25. A method for preventing or treating a cancer relapse or recurrence associated with the presence of Sortilin-expressing cancer stem cells (CSCs) in a subject, the method comprising administering to the subject an effective amount of the conjugate or salt thereof defined in any one of claims 1 to 24.

26. A method for treating a poor prognosis cancer associated with the presence of Sortilin-expressing cancer stem cells (CSCs) in a subject, the method comprising administering to the subject an effective amount of the conjugate or salt thereof defined in any one of claims 1 to 24.

27. A method for treating an unresectable, chemotherapy- and radiotherapy-resistant cancer associated with the presence of Sortilin-expressing cancer stem cells (CSCs) in a subject, the method comprising administering to the subject an effective amount of the conjugate or salt thereof defined in any one of claims 1 to 24.

28. The method of any one of claims 1 to 27, wherein the CSCs express at least one multidrug resistance (MDR) protein.

29. The method of claim 28, wherein the CSCs express MDR1 and/or ABCB5.

30. The method of any one of claims 1 to 29, wherein the cancer is breast cancer, urogenital cancer, pancreatic cancer, lung cancer, thyroid cancer, kidney cancer, gastrointestinal tract cancer, neuroendocrine tumors, neuroblastoma, skin cancer, brain cancer and leukemia.

31. The method of claim 30, wherein the urogenital cancer is ovarian cancer, prostate cancer, endometrial cancer or testis cancer.

32. The method of claim 30, wherein the breast cancer is invasive ductal carcinoma (IDC) or triple negative breast cancer (TNBC).

33. The method of claim 30, wherein the kidney cancer is renal cell carcinoma (RCC).

34. The method of claim 30, wherein the gastrointestinal tract cancer is colorectal cancer.

35. The method of claim 30, wherein the skin cancer is melanoma.

36. The method of claim 30, wherein the brain cancer is glioma.

37. The method of claim 30, wherein the leukemia is B cell Chronic Lymphocytic Leukemia (B-CLL).

38. The method of claim 30, wherein the cancer is breast cancer, ovarian cancer or pancreatic cancer.

39. The method of any one of claims 1 to 38, wherein the administration of the conjugate or salt thereof inhibits migration of the CSCs in the subject.

40. The method of any one of claims 1 to 39, wherein the method further comprises administering one or more additional active agents or therapies to the subject.

41. The method of claim 40, where the one or more additional active agents or therapies comprise radiotherapy, surgery, chemotherapeutic agents, immunotherapies, checkpoint inhibitors, and/or cell-based therapies.

42. Use of the conjugate or salt thereof defined in any one of claims 1 to 24 for the preparation of a medicament for treating a cancer comprising Sortilin-expressing cancer stem cells (CSCs) in a subject.

43. Use of the conjugate or salt thereof defined in any one of claims 1 to 24 for the preparation of a medicament for preventing or treating a cancer relapse or recurrence associated with the presence of Sortilin-expressing cancer stem cells (CSCs) in a subject.

44. Use of the conjugate or salt thereof defined in any one of claims 1 to 24 for the preparation of a medicament for treating a poor prognosis cancer associated with the presence of Sortilin-expressing cancer stem cells (CSCs) in a subject.

45. Use of the conjugate or salt thereof defined in any one of claims 1 to 24 for the preparation of a medicament for treating an unresectable, chemotherapy- and radiotherapy-resistant cancer associated with the presence of Sortilin-expressing cancer stem cells (CSCs) in a subject.

46. The use of any one of claims 42 to 45, wherein the CSCs express at least one multidrug resistance (MDR) protein.

47. The use of claim 46, wherein the CSCs express MDR1 and/or ABCB5.

48. The use of any one of claims 42 to 47, wherein the cancer is breast cancer, urogenital cancer, pancreatic cancer, lung cancer, thyroid cancer, kidney cancer, gastrointestinal tract cancer, neuroendocrine tumors, neuroblastoma, skin cancer, brain cancer and leukemia.

49. The use of claim 48, wherein the urogenital cancer is ovarian cancer, prostate cancer, endometrial cancer or testis cancer.

50. The use of claim 48, wherein the breast cancer is invasive ductal carcinoma (IDC) or triple negative breast cancer (TNBC).

51. The use of claim 48, wherein the kidney cancer is renal cell carcinoma (RCC).

52. The use of claim 48, wherein the gastrointestinal tract cancer is colorectal cancer.

53. The use of claim 48, wherein the skin cancer is melanoma.

54. The use of claim 48, wherein the brain cancer is glioma.

55. The use of claim 48, wherein the leukemia is B cell Chronic Lymphocytic Leukemia (B-CLL).

56. The use of claim 48, wherein the cancer is breast cancer, ovarian cancer or pancreatic cancer.

57. The use of any one of claims 42 to 56, wherein the medicament inhibits migration of the CSCs in the subject.

58. The use of any one of claims 42 to 57, wherein the medicament is for use with one or more additional active agents or therapies.

59. The use of claim 58, where the one or more additional active agents or therapies comprise radiotherapy, surgery, chemotherapeutic agents, immunotherapies, checkpoint inhibitors, and/or cell-based therapies.

60. The conjugate or salt thereof as defined in any one of claims 1 to 24 for use in treating a cancer comprising Sortilin-expressing cancer stem cells (CSCs) in a subject.

61. The conjugate or salt thereof as defined in any one of claims 1 to 24 for use in preventing or treating a cancer relapse or recurrence associated with the presence of Sortilin-expressing cancer stem cells (CSCs) in a subject.

62. The conjugate or salt thereof as defined in any one of claims 1 to 24 for use in treating a poor prognosis cancer associated with the presence of Sortilin-expressing cancer stem cells (CSCs) in a subject.

63. The conjugate or salt thereof as defined in any one of claims 1 to 24 for use in treating an unresectable, chemotherapy- and radiotherapy-resistant cancer associated with the presence of Sortilin-expressing cancer stem cells (CSCs) in a subject.

64. The conjugate or salt thereof for use according to any one of claims 60 to 63, wherein the CSCs express at least one multidrug resistance (MDR) protein.

65. The conjugate or salt thereof for use according to claim 64, wherein the CSCs express MDR1 and/or ABCB5.

66. The conjugate or salt thereof for use according to any one of claims 61 to 65, wherein the cancer is breast cancer, urogenital cancer, pancreatic cancer, lung cancer, thyroid cancer, kidney cancer, gastrointestinal tract cancer, neuroendocrine tumors, neuroblastoma, skin cancer, brain cancer and leukemia.

67. The conjugate or salt thereof for use according to claim 66, wherein the urogenital cancer is ovarian cancer, prostate cancer, endometrial cancer or testis cancer.

68. The conjugate or salt thereof for use according to claim 66, wherein the breast cancer is invasive ductal carcinoma (IDC) or triple negative breast cancer (TNBC).

69. The conjugate or salt thereof for use according to claim 66, wherein the kidney cancer is renal cell carcinoma (RCC).

70. The conjugate or salt thereof for use according to claim 66, wherein the gastrointestinal tract cancer is colorectal cancer.

71. The conjugate or salt thereof for use according to claim 66, wherein the skin cancer is melanoma.

72. The conjugate or salt thereof for use according to claim 66, wherein the brain cancer is glioma.

73. The conjugate or salt thereof for use according to claim 66, wherein the leukemia is B cell Chronic Lymphocytic Leukemia (B-CLL).

74. The conjugate or salt thereof for use according to claim 66, wherein the cancer is breast cancer, ovarian cancer or pancreatic cancer.

75. The conjugate or salt thereof for use according to any one of claims 60 to 74, wherein the conjugate or salt thereof inhibits migration of the CSCs in the subject.

76. The conjugate or salt thereof for use according to any one of claims 60 to 75, wherein the conjugate or salt thereof is for use with one or more additional active agents or therapies.

77. The conjugate or salt thereof according to claim 76, where the one or more additional active agents or therapies comprise radiotherapy, surgery, chemotherapeutic agents, immunotherapies, checkpoint inhibitors, and/or cell-based therapies.
